SEQUENCE LISTING (2 sequences)

SEQ ID NO: 1 - DNA, 22 bp, Homo sapiens
gccagcgtgcaaataatgaagg

SEQ ID NO: 2 - DNA, 21 bp, Homo sapiens
agagggggattatgtatccac

(19) United States
(12) Patent Application Publication
(10) Pub. No.: US 2008/0214536 A1
(43) Pub. Date: Sep. 4, 2008

(54) Amido-Substituted 6-Phenylphenanthridines

(75) Inventors: Ulrich Kautz, Allensbach (DE); Beate Schmidt, Allensbach (DE); Dieter Flockerzi, Allensbach (DE); Maria Vittoria Chiesa, Konstanz (IT); Armin Hatzelmann, Konstanz (DE); Christof Zitt, Konstanz (DE); Johannes Barsig, Konstanz (DE); Degenhard Marx, Moos (DE); Hans-Peter Kley, Allensbach (DE)

Correspondence Address: NATH & ASSOCIATES PLLC, 112 South West Street, Alexandria, VA, 22314, US

(73) Assignee: NYCOMED Gmbh, Konstanz (DE)

(21) Appl. No.: 11/885,425

(22) PCT Filed: Mar. 9, 2006
(86) PCT No.: PCT/EP06/60595

§ 371 Date: Aug. 31, 2007

(30) Foreign Application Priority Data
Mar. 9, 2005 (EP) 05101820.8

(51) Int. Cl. A61K 31/473; C07D 221/12; C07D 401/02; A61K 31/5377; A61P 11/00; A61K 31/497; C07D 413/02

(52) U.S. Cl. 514/232.8; 546/108; 544/361; 544/126; 514/298; 514/253.03

(57) ABSTRACT

Compounds of a certain formula (I)

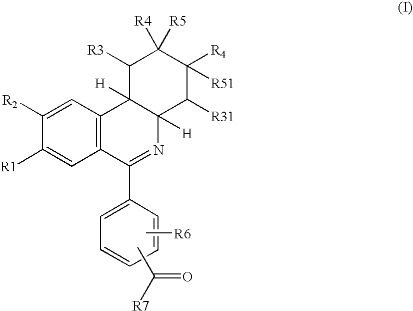

in which R1, R2, R3, R31, R4, R5, R51, R6 and R7 have the meanings indicated in the description, are novel effective PDE4 inhibitors.

### FIELD OF APPLICATION OF THE INVENTION

The invention relates to novel amido-substituted 6-phenylphenanthridine derivatives, which are used in the pharmaceutical industry for the production of pharmaceutical compositions.

### KNOWN TECHNICAL BACKGROUND

The international application WO 97/28131 discloses phenanthridines as bronchial therapeutic agents,

The international application WO 99/05113 discloses 6-phenylphenanthridines as bronchial therapeutics.

The international application WO 00/42020 discloses phenylphenanthridines with PDE4 inhibiting properties.

The international application WO 0205616 discloses phenylphenanthridines with PDE4 inhibiting properties.

The international applications WO 2004/018431 and WO 02/06238 disclose phenanthridines as PDE4 inhibitors,

The international applications WO 00/42018 and WO 00/42017 disclose phenanthridines as bronchial therapeutics.

### DESCRIPTION OF THE INVENTION

It has now been found that the novel amido-substituted 6-phenylphenanthridines described in greater detail below differ from the previously known compounds by unanticipated by unanticipated substitution patterns on the 6-phenyl ring and have surprising and particularly advantageous properties.

The invention thus relates to compounds of formula I,

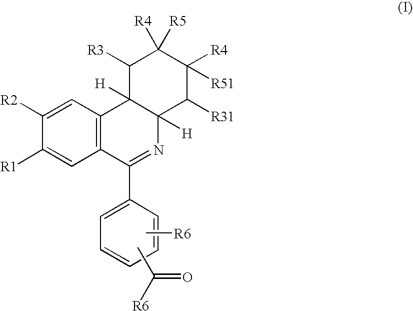

in which
R1 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
R2 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
   or in which R1 and R2 together are a 1-2C-alkylenedioxy group,
R3 is hydrogen or 1-4C-alkyl,
R31 is hydrogen or 1-4C-alkyl,
   or in which R3 and R31 together are a 1-4C-alkylene group,
R4 is hydrogen or 1-4C-alkyl,
R5 is hydrogen,
R51 is hydrogen,
   or in which R5 and R51 together represent an additional bond,
R6 is hydrogen, halogen, 1-4C-alkyl or 1-4C-alkoxy,
   either,
   in a first aspect (aspect 1) according to the present invention,
R7 is —N(R8)R9, in which
R8 is hydrogen, 1-4C-alkyl or 1-4C-alkoxy-2-4C-alkyl,
R9 is hydrogen, 1-4C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
Har1 is optionally substituted by R91 and/or R92, and is a 5- to 10-membered monocylic or fused bicyclic unsaturated heteroaryl radical comprising 1 to 4 heteroatoms selected independently from the group consisting of oxygen, nitrogen and sulfur, in which
R91 is 1-4C-alkyl or 1-4C-alkoxy,
R92 is 1-4C-alkyl or 1-4C-alkoxy,
R93 is hydrogen or 1-4C-alkyl,
R94 is hydrogen or 1-4C-alkyl,
   or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
   or R8 and R9 together and with inclusion of the nitrogen atom, to which they are attached, form a heterocyclic ring Het2, in which
Het2 is optionally substituted by R10, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is hydrogen or 1-4C-alkyl,
R13 is hydrogen or 1-4C-alkyl,
   or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
   or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is optionally substituted by R201 and/or R202, and is a 5- to 10-membered monocylic or fused bicyclic unsaturated heteroaryl radical comprising 1 to 4 heteroatoms selected independently from the group consisting of oxygen, nitrogen and sulfur, in which
R201 is 1-4C-alkyl or 1-4C-alkoxy,
R202 is 1-4C-alkyl or 1-4C-alkoxy,
Het6 is optionally substituted by R203 and/or R204, and is a monocylic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy
R206 is 1-4C-alkoxy
R21 is aminosulphonyl,
   or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which
R181 is 1-4C-alkyl,
   under the provisio, that those compounds, in which
R1 is hydroxyl, 1-4C-alkoxy, 3-7C-cycloalkoxy, 3-7C-cycloalkylmethoxy, or completely or predominantly fluorine-substituted 1-4C-alkoxy,
R2 is hydroxyl, 1-4C-alkoxy, 3-7C-cycloalkoxy, 3-7C-cycloalkylmethoxy, or completely or predominantly fluorine-substituted 1-4C-alkoxy,
   or in which R1 and R2 together are a 1-2C-alkylenedioxy group,
   and
R7 is —N(R8)R9, in which
   either
R8 is hydrogen or 1-4C-alkyl, and
R9 is hydrogen, 1-4C-alkyl or 3-7C-cycloalkyl,
   or
R8 is hydrogen or 1-4C-alkyl, and
R9 is pyridyl optionally substituted by 1-4C-alkyl or 1-4C-alkoxy,
   or
   or R8 and R9 together and with inclusion of the nitrogen atom, to which they are attached, form a heterocyclic ring Het2, in which
Het2 is pyrrolidin-1-yl, piperidin-1-yl, piperazin-1-yl, 4-methyl-piperazin-1-yl or morpholin-4-yl,
   are thereof disclaimed,
   and the salts, the N-oxides and the salts of the N-oxides of these compounds.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and preferably the ethyl and methyl radicals.

2-4C-Alkyl represents a straight-chain or branched alkyl radical having 2 to 4 carbon atoms. Examples which may be mentioned are the butyl, isobutyl, sec-butyl, tert-butyl, propyl, isopropyl and preferably the ethyl radical.

3-7C-Cycloalkyl represents cyclopropyl, cyclobutyl, cyclopentyl, cyclohexyl and cycloheptyl, of which cyclopropyl, cyclobutyl and cyclopentyl are preferred.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

3-7C-Cycloalkoxy represents cyclopropyloxy, cyclobutyloxy, cyclopentyloxy, cyclohexyloxy and cycloheptyloxy, of which cyclopropyloxy, cyclobutyloxy and cyclopentyloxy are preferred.

3-7C-Cycloalkylmethoxy represents cyclopropylmethoxy, cyclobutylmethoxy, cyclopentylmethoxy, cyclohexylmethoxy and cycloheptylmethoxy, of which cyclopropylmethoxy, cyclobutylmethoxy and cyclopentylmethoxy are preferred.

As completely or predominantly fluorine-substituted 1-4C-alkoxy, for example, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals may be mentioned. “Predominantly” in this connection means that more than half of the hydrogen atoms of the 1-4C-alkoxy radicals are replaced by fluorine atoms.

1-2C-Alkylenedioxy represents, for example, the methylenedioxy [—O—CH₂—O—] and the ethylenedioxy [—O—CH₂—CH₂—O—] radicals.

1-4C-Alkoxy-2-4C-alkyl represents one of the abovementioned 2-4C-alkyl radicals, which is substituted by one of the abovementioned 1-4C-alkoxy radicals. Examples which may be mentioned are the methoxyethyl and the isopropoxyethyl radicals, particularly the 2-methoxyethyl and the 2-isopropoxyethyl radicals.

Hydroxy-2-4C-alkyl represents 2-4C-alkyl radicals, which are substituted by a hydroxyl group. Examples which may be mentioned are the 2-hydroxyethyl and the 3-hydroxypropyl radicals.

1-4C-Alkoxycarbonyl represents a radical which, in addition to the carbonyl group, contains one of the abovementioned 1-4C-alkoxy radicals. Examples which may be mentioned are the methoxycarbonyl, the ethoxycarbonyl and the isopropoxycarbonyl radicals.

Halogen within the meaning of the invention is bromine, chlorine or fluorine.

Pyridinyl-1-4C-alkyl represents one of the abovementioned 1-4C-alkyl radicals, which is substituted by a pyridyl radical. Examples which may be mentioned are the pyridylmethyl, the 2-pyridylethyl and the 3-pyridylpropyl radicals.

Pyridinyl or pyridyl includes pyridin-2-yl, pyridin-3-yl and pyridin-4-yl.

Aryl-1-4C-alkyl represents one of the abovementioned 1-4C-alkyl radicals, which is substituted by an aryl radical. Examples which may be mentioned are the arylmethyl, the 2-arylethyl and the 3-arylpropyl radicals.

Aryl stands for R205- and/or R206-substituted phenyl.

Mono- or di-1-4C-alkoxy-2-4C-alkyl represents 2-4C-alkyl radicals, which are substituted by one or two of the abovementioned 1-4C-alkoxy radicals. Examples which may be mentioned are the methoxyethyl, ethoxyethyl and the isopropoxyethyl radicals, particularly the 2-methoxyethyl, 2-ethoxyethyl and the 2-isopropoxyethyl radicals, as well as the dimethoxy-ethyl and the diethoxy-ethyl radicals, particularly the 2,2-dimethoxy-ethyl and the 2,2-diethoxy-ethyl radicals.

Mono- or di-1-4C-alkoxycarbonyl-1-4C-alkyl represents one of the abovementioned 1-4C-alkyl radicals, which is substituted by one or two of the abovementioned 1-4C-alkoxycarbonyl radicals. Examples which may be mentioned are the methoxycarbonylmethyl, the 2-methoxycarbonylethyl and the 1,2-(dimethoxycarbonyl)-ethyl radicals.

Each of the radicals Het1, Het2, Het3, Het4, Het5 and Het7 is optionally substituted as indicated above, and represents independently a 3- to 7-membered fully saturated monocyclic heterocyclic ring radical comprising one nitrogen atom as indicated above and optionally one further heteroatom selected from the group consisting of oxygen, nitrogen and sulfur.

Het1, Het2, Het3, Het4, Het5 and Het7 may include independently, without being restricted thereto, aziridinyl, azetidinyl, pyrrolidinyl, piperidinyl, homopiperidinyl, morpholinyl, thiomorpholinyl, oxazolidinyl, isoxazolidinyl, thiazolidinyl, isothiazolidinyl, pyrazolidinyl, imidazolidinyl, piperazinyl or homopiperazinyl.

As further examples for Het1, Het2, Het3, Het4, Het5 or Het7 according to this invention may be mentioned, without being restricted thereto, derivatives of the abovementioned exemplary radicals which are substituted by a substituent as indicated above, notably, for example, those radicals, which are substituted on a ring nitrogen atom by a substituent as indicated above, such as, as example for Het2, 4-N—(R10)-piperazinyl or 4-N—(R10)-homopiperazinyl, or, as example for Het7, 4-N—(R181)-piperazinyl or 4-N—(R181)-homopiperazinyl.

Illustratively, a suitable example for Het1, Het2, Het3, Het4, Het5 and Het7 radicals include, for example, without being restricted thereto, morpholin-4-yl. Further suitable examples include for Het2, without being restricted thereto, 4-N—(R10)-piperazin-1-yl, and for Het7, without being restricted thereto, 4-N—(R181)-piperazin-1-yl.

Het6 is optionally substituted by R203 and/or R204 and stands for a monocylic 3- to 7-membered fully saturated heterocyclic ring radical comprising one to three heteroatoms, each of which is selected from the group consisting of nitrogen, oxygen and sulfur.

In particular, Het6 is optionally substituted by R203 and/or R204 and refers within the meaning of this invention, in a special facet (facet 1) according to the present invention, to a monocyclic 3- to 7-membered fully saturated heterocyclic ring radical comprising one nitrogen atom and optionally one further heteroatom selected from the group consisting of oxygen, nitrogen and sulfur.

More precisely, within the context of this invention, Het6 can be bonded to the carbonyl moiety of —C(O)R20, in one facet (facet 1a) of this invention, via a ring carbon atom or, in another facet (facet 1a′), via a ring nitrogen atom.

Yet more precisely, Het6 is optionally substituted by R203 and/or R204 on a ring nitrogen or ring carbon atom.

Het6 may include, without being restricted thereto, aziridinyl, azetidinyl, pyrrolidinyl, piperidinyl, homopiperidinyl, morpholinyl, thiomorpholinyl, oxazolidinyl, isoxazolidinyl, thiazolidinyl, isothiazolidinyl, pyrazolidinyl, imidazolidinyl, piperazinyl or homopiperazinyl.

In detailed example, Het6 may include according to facet 1a, without being restricted thereto, piperazin-2-yl, piperidin-3-yl, morpholin-3-yl or piperidin-4-yl.

Furthermore in detailed example, Het6 may include according to facet 1a′, without being restricted thereto, aziridin-1-yl, azetidin-1-yl, pyrrolidin-1-yl, piperidin-1-yl, homopiperidin-1-yl, pyrazolidin-1-yl, piperazin-1-yl, homopiperazin-1-yl, morpholin-4-yl or thiomorpholin-4-yl.

As further examples for Het6 according to this invention may be mentioned, without being restricted thereto, R203- and/or R204-substituted derivatives of the abovementioned exemplary Het6 radicals, such as, for example according to facet 1a, 1-N—(R203)-4-N—(R204)-piperazin-2-yl, or according to facet 1a′, 4-N—(R203)-piperazin-1-yl.

Illustratively, as exemplary suitable Het6 radicals may be mentioned, for example, without being restricted thereto, morpholin-4-yl or 1-N—(R203)-4-N—(R204)-piperazin-2-yl.

Har1 is optionally substituted by R91 and/or R92, and is a 5- to 10-membered monocylic or fused bicyclic unsaturated (heteroaromatic) heteroaryl radical comprising 1 to 4 heteroatoms selected independently from the group consisting of oxygen, nitrogen and sulfur.

It is to be understood that the radical Har1 is bonded to the parent molecular group via a ring carbon atom.

Har1 may include, without being restricted thereto, furanyl, thiophenyl, pyrrolyl, oxazolyl, isoxazolyl, thiazolyl, isothiazolyl, imidazolyl, pyrazolyl, triazolyl (precisely: 1,2,4-triazolyl or 1,2,3-triazolyl), thiadiazolyl (precisely: 1,3,4-thiadiazolyl, 1,2,5-thiadiazolyl, 1,2,3-thiadiazolyl or 1,2,4-thiadiazolyl), oxadiazolyl (precisely: 1,3,4-oxadiazolyl, 1,2,5-oxadiazolyl, 1,2,3-oxadiazolyl or 1,2,4-oxadiazolyl) or tetrazolyl; or, pyridinyl, pyrimidinyl, pyrazinyl or pyridazinyl; or the fused or benzofused derivatives of the abovementioned exemplary radicals, such as, for example those mentioned more detailed in the embodimental detail below; as well as the R91- and/or R92-substituted derivatives of theses radicals.

In an embodimental detail (detail 1) according to this invention, Har1 is optionally substituted by R91 and/or R92, and is a 9- or 10-membered fused bicyclic unsaturated (heteroaromatic) heteroaryl radical comprising 1 to 4 heteroatoms independently selected from the group consisting of oxygen, nitrogen and sulfur.

Har1 may include according to this detail 1, without being restricted thereto, benzothiophenyl, benzofuranyl, indolyl, benzoxazolyl, benzothiazolyl, indazolyl, benzimidazolyl, benzisoxazolyl, benzisothiazolyl, benzofurazanyl, benzotriazolyl, benzothiadiazolyl, quinolinyl, isoquinolinyl, quinazolinyl, quinoxalinyl, phthalazinyl or cinnolinyl; or indolizinyl, purinyl, naphthyridinyl, imidazopyridinyl or pteridinyl; as well as the R91- and/or R92-substituted derivatives thereof.

Illustratively, as exemplary suitable Har1 radicals according to detail 1 may be mentioned, for example, without being restricted thereto, quinolinyl, naphthyridinyl or imidazopyridinyl, as well as the R91- and/or R92-substituted derivatives thereof.

As more specific exemplary suitable Har1 radicals according to detail 1 may be mentioned, for example, without being restricted thereto, quinolin-3-yl, 2,3-dimethyl-imidazo[1,2-a]pyridin-7-yl or [1,7]naphthyridin-8-yl.

In a further embodimental detail (detail 2) according to this invention, Har1 is optionally substituted by R91 and/or R92, and is a 6-membered monocyclic unsaturated (heteroaromatic) heteroaryl radical comprising one or two nitrogen atoms.

Har1 may include according to this detail 2, without being restricted thereto, pyridinyl, pyrimidinyl, pyrazinyl or pyridazinyl; as well as the R91- and/or R92-substituted derivatives thereof.

Illustratively, as exemplary suitable Har1 radicals according to detail 2 may be mentioned, for example, without being restricted thereto, pyridinyl, as well as the R91- and/or R92-substituted derivatives thereof.

As more specific exemplary suitable Har1 radicals according to detail 2 may be mentioned, for example, without being restricted thereto, dimethoxypyridinyl, such as, for example, 2,6-dimethoxypyridin-4-yl or, in particular, 2,6-dimethoxypyridin-3-yl.

Har2 is optionally substituted by R201 and/or R202, and is a 5- to 10-membered monocylic or fused bicyclic unsaturated (heteroaromatic) heteroaryl radical comprising 1 to 4 heteroatoms selected independently from the group consisting of oxygen, nitrogen and sulfur.

Preferably, the radical Har2 is bonded to the parent molecular group via a ring carbon atom.

Har2 may include, without being restricted thereto, furanyl, thiophenyl, pyrrolyl, oxazolyl, isoxazolyl, thiazolyl, isothiazolyl, imidazolyl, pyrazolyl, triazolyl (precisely: 1,2,4-triazolyl or 1,2,3-triazolyl), thiadiazolyl (precisely: 1,3,4-thiadiazolyl, 1,2,5-thiadiazolyl, 1,2,3-thiadiazolyl or 1,2,4-thiadiazolyl), oxadiazolyl (precisely: 1,3,4-oxadiazolyl, 1,2,5-oxadiazolyl, 1,2,3-oxadiazolyl or 1,2,4-oxadiazolyl) or tetrazolyl; or, pyridinyl, pyrimidinyl, pyrazinyl or pyridazinyl; or the fused or benzofused or pyridofused derivatives of the abovementioned exemplary radicals; as well as the R201- and/or R202-substituted derivatives of theses radicals.

In an embodimental detail according to this invention, Har2 is optionally substituted by R201 and/or R202, and is a 6-membered monocyclic unsaturated (heteroaromatic) heteroaryl radical comprising one or two nitrogen atoms.

Har2 may include according to this detail, without being restricted thereto, pyridinyl, pyrimidinyl, pyrazinyl or pyridazinyl; as well as the R201- and/or R202-substituted derivatives thereof.

Illustratively, as exemplary suitable Har2 radical may be mentioned, for example, without being restricted thereto, pyridinyl.

As more specific exemplary suitable Har2 radicals may be mentioned, for example, without being restricted thereto, pyridin-3-yl or pyridin-4-yl.

The heterocyclic groups mentioned herein refer, unless otherwise mentioned, to all of the possible isomeric forms thereof.

The heterocyclic groups mentioned herein refer, unless otherwise noted, in particular to all of the possible positional isomers thereof.

Thus, for example, the term pyridyl or pyridinyl includes pyridin-2-yl, pyridin-3-yl and pyridin-4-yl.

The heterocyclic groups mentioned herein refer, unless otherwise noted, also to all of the possible tautomers thereof, in pure form as well as any mixtures thereof.

Constituents which are optionally substituted as stated herein, may be substituted, unless otherwise noted, at any possible position.

The heterocyclic groups, alone or as part of other groups, mentioned herein may be substituted by their given substituents, unless otherwise noted, at any possible position, such as e.g. at any substitutable ring carbon or ring nitrogen atom.

Unless otherwise noted, rings containing quaternizable imino-type ring nitrogen atoms (—N═) may be preferably not quaternized on these imino-type ring nitrogen atoms by the mentioned substituents or parent molecular groups.

Unless otherwise noted, any heteroatom of a heterocyclic ring with unsatisfied valences mentioned herein is assumed to have the hydrogen atom(s) to satisfy the valences.

When any variable occurs more than one time in any constituent, each definition is independent.

As it is known for the person skilled in the art, compounds comprising nitrogen atoms can form N-oxides. Particularly, imine nitrogen, especially heterocyclic or heteroaromatic imine nitrogen, or pyridine-type nitrogen (═N—) atoms, can be N-oxidized to form the N-oxides comprising the group ═N⁺(O⁻)—. Thus, the compounds according to the present invention comprising the imine nitrogen atom in position 5 of the phenylphenanthridine backbone and, optionally (depending on the meaning of R7), one or more further nitrogen atoms suitable to exist in the N-oxide state (═N⁺(O⁻)—) may be capable to form (depending on the number of nitrogen atoms suitable to form stabile N-oxides) mono-N-oxides, bis-N-oxides or multi-N-oxides, or mixtures thereof.

The term N-oxide(s) as used in this invention therefore encompasses all possible, and in particular all stabile, N-oxide forms, such as mono-N-oxides, bis-N-oxides or multi-N-oxides, or mixtures thereof in any mixing ratio.

Possible salts for compounds of the formula I—depending on substitution—are all acid addition salts or all salts with bases. Particular mention may be made of the pharmacologically tolerable salts of the inorganic and organic acids and bases customarily used in pharmacy. Those suitable are, on the one hand, water-insoluble and, particularly, water-soluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic add, it being possible to employ the acids in salt preparation—depending on whether a mono- or polybasic add is concerned and depending on which salt is desired—in an equimolar quantitative ratio or one differing therefrom.

On the other hand, salts with bases are also suitable. Examples of salts with bases which may be mentioned are alkali metal (lithium, sodium, potassium) or calcium, aluminum, magnesium, titanium, ammonium, meglumine or guanidinium salts, where here too the bases are employed in salt preparation in an equimolar quantitative ratio or one differing therefrom.

Pharmacologically intolerable salts which can initially be obtained, for example, as process products in the preparation of the compounds according to the invention on an industrial scale are converted into pharmacologically tolerable salts by processes known to the person skilled in the art.

It is known to the person skilled in the art that the compounds of formula I according to the invention and their salts, when they are isolated, for example, in crystalline form, can contain various amounts of solvents. The invention therefore also comprises all solvates and in particular all hydrates of the compounds of the formula I, and also all solvates and in particular all hydrates of the salts of the compounds of the formula I.

The substituents R6 and —C(O)R7 of compounds of formula I can be attached in the ortho, meta or para position with respect to the binding position in which the 6-phenyl ring is bonded to the phenanthridine ring system, whereby preference is given to the attachement of —C(O)R7 in the meta or in the para position. In another embodiment preference is given to the attachement of —C(O)R7 in the meta or in the para position, and R6 is hydrogen. In yet another embodiment preference is given to the attachement of —C(O)R7 in the meta position, and R6 is hydrogen. In still yet another embodiment preference is given to the attachement of —C(O)R7 in the para position, and R6 is hydrogen.

Compounds of formula I to be more worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3, R31, R4, R5, R51 and R6 are hydrogen,
   either,
   in a first aspect (aspect 1) according to the present invention,
R7 is —N(R8)R9, in which
R8 is hydrogen, 1-4C-alkyl or 1-4C-alkoxy-2-4C-alkyl,
R9 is hydrogen, 1-4C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
Har1 is optionally substituted by R91 and/or R92, and is a 5- to 10-membered monocylic or fused bicyclic unsaturated heteroaryl radical comprising 1 to 4 heteroatoms selected independently from the group consisting of oxygen, nitrogen and sulfur, in which
R91 is 1-4C-alkyl or 1-4C-alkoxy,
R92 is 1-4C-alkyl or 1-4C-alkoxy,
R93 is hydrogen or 1-4C-alkyl,
R94 is hydrogen or 1-4C-alkyl,
   or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
   or R8 and R9 together and with inclusion of the nitrogen atom, to which they are attached, form a heterocyclic ring Het2, in which
Het2 is optionally substituted by R10, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is hydrogen or 1-4C-alkyl,
R13 is hydrogen or 1-4C-alkyl,
   or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
   or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is optionally substituted by R201 and/or R202, and is a 5- to 10-membered monocylic or fused bicyclic unsaturated heteroaryl radical comprising 1 to 4 heteroatoms selected independently from the group consisting of oxygen, nitrogen and sulfur, in which
R201 is 1-4C-alkyl or 1-4C-alkoxy,
R202 is 1-4C-alkyl or 1-4C-alkoxy,
Het6 is optionally substituted by R203 and/or R204, and is a monocylic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy
R206 is 1-4C-alkoxy
R21 is aminosulphonyl,
   or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which
R181 is 1-4C-alkyl,
   under the provisio, that those compounds, in which
R1 is 1-2C-alkoxy, or completely or predominantly fluorine-substituted 1-2C-alkoxy,
R2 is 1-2C-alkoxy, or completely or predominantly fluorine-substituted 1-2C-alkoxy,
   and
R7 is —N(R8)R9, in which
   either
R8 is hydrogen or 1-4C-alkyl, and
R9 is hydrogen, 1-4C-alkyl or 3-7C-cycloalkyl,
   or
R8 is hydrogen or 1-4C-alkyl, and
R9 is pyridyl optionally substituted by 1-4C-alkyl or 1-4C-alkoxy,
   or
   or R8 and R9 together and with inclusion of the nitrogen atom, to which they are attached, form a heterocyclic ring Het2, in which
Het2 is pyrrolidin-1-yl, piperidin-1-yl, piperazin-1-yl, 4-methyl-piperazin-1-yl or morpholin-4-yl,
   are thereof disclaimed,
   and the salts, the N-oxides and the salts of the N-oxides of these compounds.

Compounds of formula I in particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3, R31, R4, R5, R51 and R6 are hydrogen,
   either,
   in a first aspect (aspect 1) according to the present invention,
R7 is —N(R8)R9, in which
R8 is hydrogen, 1-4C-alkyl or 1-4C-alkoxy-2-4C-alkyl,
R9 is hydrogen, 1-4C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
   either
Har1 is optionally substituted by R91 and/or R92, and is a 9- or 10-membered fused bicyclic unsaturated heteroaryl radical comprising 1 to 4 heteroatoms selected independently from the group consisting of oxygen, nitrogen and sulfur, in which
R91 is 1-4C-alkyl,
R92 is 1-4C-alkyl,
   or
Har1 is optionally substituted by R91 and/or R92, and is a 6-membered monocyclic unsaturated heteroaryl radical comprising one or two nitrogen atoms, in which
R91 is 1-4C-alkoxy,
R92 is 1-4C-alkoxy,
R93 is hydrogen or 1-4C-alkyl,
R94 is hydrogen or 1-4C-alkyl,
   or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
   or R8 and R9 together and with inclusion of the nitrogen atom, to which they are attached, form a heterocylic ring Het2, in which
Het2 is optionally substituted by R10, and is a 3- to 7-membered saturated monocyclic heterocylic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is hydrogen or 1-4C-alkyl,
R13 is hydrogen or 1-4C-alkyl,
   or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
   or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is a 6-membered monocylic unsaturated heteroaryl radical comprising one or two nitrogen atoms,
Het6 is optionally substituted by R203 and/or R204, and is a monocylic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy
R206 is 1-4C-alkoxy
R21 is aminosulphonyl,
   or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which
R181 is 1-4C-alkyl,
   under the provisio, that those compounds, in which
R1 is 1-2C-alkoxy, or completely or predominantly fluorine-substituted 1-2C-alkoxy,
R2 is 1-2C-alkoxy, or completely or predominantly fluorine-substituted 1-2C-alkoxy,
   and
R7 is —N(R8)R9, in which
   either
R8 is hydrogen or 1-4C-alkyl, and
R9 is hydrogen, 1-4C-alkyl or 3-7C-cycloalkyl,
   or
R8 is hydrogen or 1-4C-alkyl, and
R9 is pyridyl optionally substituted by 1-4C-alkoxy,
   or
   or R8 and R9 together and with inclusion of the nitrogen atom, to which they are attached, form a heterocyclic ring Het2, in which
Het2 is pyrrolidin-1-yl, piperidin-1-yl, piperazin-1-yl, 4-methyl-piperazin-1-yl or morpholin-4-yl,
   are thereof disclaimed,
   and the salts, the N-oxides and the salts of the N-oxides of these compounds.

Compounds of formula I in more particular worthy to be mentioned are those in which
one of R1 and R2 is methoxy, and the other is methoxy, ethoxy, 2,2-difluoroethoxy, or difluoromethoxy,
R3, R31, R4, R5, R51 and R6 are hydrogen,
   either,
   in a first aspect (aspect 1) according to the present invention,
R7 is —N(R8)R9, in which
R8 is hydrogen, 1-4C-alkyl or 1-4C-alkoxy-2-4C-alkyl,
R9 is 1-4C-alkyl, mono- or di-1-4C-alkoxy-2-4C-alkyl, hydroxy-2-4C-alkyl, mono- or di-1-2C-alkoxycarbonyl-1-4C-alkyl, Har1, pyridinyl-1-4C-alkyl, 3-5C-cycloalkyl, or 2-4C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl, R93 and R94 together and with inclusion of the nitrogen atom, to which they are attached, form a heterocyclic ring Het1, in which
Het1 is morpholinyl,
   or R8 and R9 together and with inclusion of the nitrogen atom, to which they are attached, form a heterocyclic ring Het2, in which
Het2 is pyrrolidinyl, morpholinyl or 4N—(R10)-piperazinyl, in which
R10 is —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is 1-4C-alkyl,
R13 is 1-4C-alkyl,
   or R12 and R13 together and with inclusion of the nitrogen atom, to which they are attached, form a heterocyclic ring Het3, in which
Het3 is morpholinyl,
R14 is 1-4C-alkyl,
R15 is 1-4C-alkyl,
   or R14 and R15 together and with inclusion of the nitrogen atom, to which they are attached, form a heterocyclic ring Het4, in which
Het4 is morpholinyl,
R16 is 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is pyridinyl, morpholinyl, 1N—(R203)₄N—(R204)-piperazinyl, or Aryl-1-2C-alkyl, in which
R203 is 1-4C-alkyl,
R204 is 1-4C-alkyl,
Aryl is 3,4-dimethoxyphenyl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is 1-4C-alkyl,
   under the provisio, that those compounds, in which
one of R1 and R2 is methoxy, and the other is methoxy, ethoxy or difluoromethoxy,
   and
R7 is —N(R8)R9, in which
   either
R8 is hydrogen or 1-4C-alkyl, and
R9 is 1-4C-alkyl or 3-5C-cycloalkyl,
   or
   or R8 and R9 together and with inclusion of the nitrogen atom, to which they are attached, form a heterocyclic ring Het2, in which
Het2 is pyrrolidin-1-yl or morpholin-4-yl,
   are thereof disclaimed,
   and the salts, the N-oxides and the salts of the N-oxides of these compounds.

Yet compounds of formula I in more particular worthy to be mentioned are those in which
one of R1 and R2 is methoxy, and the other is methoxy, ethoxy, 2,2-difluoroethoxy, or difluoromethoxy,
R3, R31, R4, R5, R51 and R6 are hydrogen,
   either,
   in a first aspect (aspect 1) according to the present invention,
R7 is —N(R8)R9, in which
R8 is hydrogen, 1-4C-alkyl or 1-4C-alkoxy-2-4C-alkyl,
R9 is mono- or di-1-4C-alkoxy-2-4C-alkyl, hydroxy-2-4C-alkyl, mono- or di-1-2C-alkoxycarbonyl-1-4C-alkyl, Har1, pyridinyl-1-4C-alkyl, or 2-4C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl, R93 and R94 together and with inclusion of the nitrogen atom, to which they are attached, form a heterocyclic ring Het1, in which
Het1 is morpholinyl,
   or R8 and R9 together and with inclusion of the nitrogen atom, to which they are attached, form a heterocyclic ring Het2, in which
Het2 is 4N—(R10)-piperazinyl, in which
R10 is —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is 1-4C-alkyl,
R13 is 1-4C-alkyl,
   or R12 and R13 together and with inclusion of the nitrogen atom, to which they are attached, form a heterocyclic ring Het3, in which
Het3 is morpholinyl,
R14 is 1-4C-alkyl,
R15 is 1-4C-alkyl,
   or R14 and R15 together and with inclusion of the nitrogen atom, to which they are attached, form a heterocyclic ring Het4, in which
Het4 is morpholinyl,
R16 is 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is pyridinyl, morpholinyl, 1N—(R203)-4-N—(R204)-piperazinyl, or Aryl-1-2C-alkyl, in which
R203 is 1-4C-alkyl,
R204 is 1-4C-alkyl,
Aryl is 3,4-dimethoxyphenyl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is 1-4C-alkyl,
   and the salts, the N-oxides and the salts of the N-oxides of these compounds.

Still yet compounds of formula I in more particular worthy to be mentioned are those in which
one of R1 and R2 is methoxy, and the other is methoxy, ethoxy, 2,2-difluoroethoxy, or difluoromethoxy,
R3, R31, R4, R5, R51 and R6 are hydrogen,
   either,
   in a first aspect (aspect 1) according to the present invention,
R7 is —N(R8)R9, in which
R8 is hydrogen, 1-4C-alkyl or 1-4C-alkoxy-2-4C-alkyl,
R9 is mono- or di-1-4C-alkoxy-2-4C-alkyl, hydroxy-2-4C-alkyl, mono- or di-1-2C-alkoxycarbonyl-1-4C-alkyl, Har1, pyridinyl-1-4C-alkyl, or 2-4C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl, R93 and R94 together and with inclusion of the nitrogen atom, to which they are attached, form a heterocyclic ring Het1, in which
Het1 is morpholinyl or 4N-(1-4C-alkyl)-piperazinyl,
   or R8 and R9 together and with inclusion of the nitrogen atom, to which they are attached, form a heterocyclic ring Het2, in which
Het2 is 4N—(R10)-piperazinyl, in which
R10 is —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is 1-4C-alkyl,
R13 is 1-4C-alkyl,
   or R12 and R13 together and with inclusion of the nitrogen atom, to which they are attached, form a heterocyclic ring Het3, in which
Het3 is morpholinyl,
R14 is 1-4C-alkyl,
R15 is 1-4C-alkyl,
   or R14 and R15 together and with inclusion of the nitrogen atom, to which they are attached, form a heterocyclic ring Het4, in which
Het4 is morpholinyl,
R16 is 1-4C-alkyl or pyridyl,
R17 is hydrogen or 1-4C-alkyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is pyridinyl, morpholinyl, 1N—(R203)-4N—(R204)-piperazinyl, or Aryl-1-2C-alkyl, in which
R203 is 1-4C-alkyl,
R204 is 1-4C-alkyl,
Aryl is 3,4-dimethoxyphenyl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is 1-4C-alkyl,
   and the salts, the N-oxides and the salts of the N-oxides of these compounds.

Compounds of formula I in further more particular worthy to be mentioned are those in which
R1 is methoxy,
R2 is methoxy, ethoxy, 2,2-difluoroethoxy, or difluoromethoxy,
R3, R31, R4, R5, R51 and R6 are hydrogen,
   either,
   in a first aspect (aspect 1) according to the present invention,
R7 is —N(R8)R9, in which
R8 is hydrogen, methyl, ethyl, isopropyl or 2-methoxyethyl,
R9 is methyl, isopropyl, methoxy-2-4C-alkyl, 2,2-diethoxyethyl, 3-hydroxy-propyl, methoxycarbonylmethyl, 1,2-di-(methoxycarbonyl)-ethyl, Har1, 2-pyridinyl-ethyl, cyclopropyl, cyclobutyl, or 2-3C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl, R93 and R94 together and with inclusion of the nitrogen atom, to which they are attached, form a heterocyclic ring Het1, in which
Het1 is morpholinyl,
   or R8 and R9 together and with inclusion of the nitrogen atom, to which they are attached, form a heterocyclic ring Het2, in which
Het2 is pyrrolidinyl, morpholinyl or 4N—(R10)-piperazinyl, in which
R10 is —C(O)R11, pyridyl, ethyl substituted by —NR(14)R15, or methyl substituted by —C(O)N(R16)R17, in which
R11 is methyl substituted by —NR(12)R13, in which
R12 is methyl,
R13 is methyl,
   or R12 and R13 together and with inclusion of the nitrogen atom, to which they are attached, form a heterocyclic ring Het3, in which
Het3 is morpholinyl,
R14 is methyl,
R15 is methyl,
   or R14 and R15 together and with inclusion of the nitrogen atom, to which they are attached, form a heterocyclic ring Het4, in which
Het4 is morpholinyl,
R16 is methyl or pyridyl,
R17 is hydrogen or methyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is pyridinyl, morpholin-4-yl, 1N—(R203)-4N—(R204)-piperazinyl, or Aryl-methyl, in which
R203 is methyl,
R204 is methyl,
Aryl is 3,4-dimethoxyphenyl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is methyl,
whereby —C(O)R7 is bonded in the meta or para position with respect to the binding position, in which the phenyl ring is attached to the phenanthridine ring system,
   under the provisio, that those compounds, in which
R1 is methoxy,
R2 is methoxy, ethoxy or difluoromethoxy,
   and
R7 is —N(R8)R9, in which
   either
R8 is hydrogen, methyl, ethyl or isopropyl, and
R9 is methyl, isopropyl, cyclopropyl or cyclobutyl,
   or
   or R8 and R9 together and with inclusion of the nitrogen atom, to which they are attached, form a heterocyclic ring Het2, in which
Het2 is pyrrolidin-1-yl or morpholin-4-yl,
   are thereof disclaimed,
   and the salts, the N-oxides and the salts of the N-oxides of these compounds.

Yet compounds of formula I in further more particular worthy to be mentioned are those in which
R1 is methoxy,
R2 is methoxy, ethoxy, 2,2-difluoroethoxy, or difluoromethoxy,
R3, R31, R4, R5, R51 and R6 are hydrogen,
   either,
   in a first aspect (aspect 1) according to the present invention,
R7 is —N(R8)R9, in which
R8 is hydrogen, methyl, ethyl, isopropyl or 2-methoxyethyl,
R9 is methoxy-2-4C-alkyl, 2,2-diethoxyethyl, 3-hydroxy-propyl, methoxycarbonylmethyl, 1,2-di(methoxycarbonyl)-ethyl, Har1, 2-pyridinyl-ethyl, or 2-3C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl, R93 and R94 together and with inclusion of the nitrogen atom, to which they are attached, form a heterocyclic ring Het1, in which
Het1 is morpholinyl,
   or R8 and R9 together and with inclusion of the nitrogen atom, to which they are attached, form a heterocyclic ring Het2, in which
Het2 is 4N—(R10)-piperazinyl, in which
R10 is —C(O)R11, pyridyl, ethyl substituted by —NR(14)R15, or methyl substituted by —C(O)N(R16)R17, in which
R11 is methyl substituted by —NR(12)R13, in which
R12 is methyl,
R13 is methyl,
   or R12 and R13 together and with inclusion of the nitrogen atom, to which they are attached, form a heterocyclic ring Het3, in which
Het3 is morpholinyl,
R14 is methyl,
R15 is methyl,
   or R14 and R15 together and with inclusion of the nitrogen atom, to which they are attached, form a heterocyclic ring Het4, in which
Het4 is morpholinyl,
R16 is methyl or pyridyl,
R17 is hydrogen or methyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is pyridinyl, morpholin-4-yl, 1N—(R203)-4N—(R204)-piperazinyl, or Aryl-methyl, in which
R203 is methyl,
R204 is methyl,
Aryl is 3,4-dimethoxyphenyl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is methyl,
whereby —C(O)R7 is bonded in the meta or para position with respect to the binding position, in which the phenyl ring is attached to the phenanthridine ring system,
   and the salts, the N-oxides and the salts of the N-oxides of these compounds.

Still yet compounds of formula I in further more particular worthy to be mentioned are those in which
R1 is methoxy,
R2 is methoxy, ethoxy, 2,2-difluoroethoxy, or difluoromethoxy,
R3, R31, R4, R5, R51 and R6 are hydrogen,
   either,
   in a first aspect (aspect 1) according to the present invention,
R7 is —N(R8)R9, in which
R8 is hydrogen, methyl, ethyl, isopropyl or 2-methoxyethyl,
R9 is methoxy-2-3C-alkyl, 2,2-diethoxyethyl, 3-hydroxy-propyl, methoxycarbonylmethyl, 1,2-di(methoxycarbonyl)-ethyl, Har1, 2-pyridinyl-ethyl, or 2-3C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl, R93 and R94 together and with inclusion of the nitrogen atom, to which they are attached, form a heterocyclic ring Het1, in which
Het1 is morpholinyl or 4N-(1-2C-alkyl)-piperazinyl,
   or R8 and R9 together and with inclusion of the nitrogen atom, to which they are attached, form a heterocyclic ring Het2, in which
Het2 is 4N—(R10)-piperazinyl, in which
R10 is —C(O)R11, pyridyl, ethyl substituted by —NR(14)R15, or methyl substituted by —C(O)N(R16)R17, in which
R11 is methyl substituted by —NR(12)R13, in which
R12 is methyl,
R13 is methyl,
   or R12 and R13 together and with inclusion of the nitrogen atom, to which they are attached, form a heterocyclic ring Het3, in which
Het3 is morpholinyl,
R14 is methyl,
R15 is methyl,
   or R14 and R15 together and with inclusion of the nitrogen atom, to which they are attached, form a heterocyclic ring Het4, in which
Het4 is morpholinyl,
R16 is methyl or pyridyl,
R17 is hydrogen or methyl,
   or R16 and R17 together and with inclusion of the nitrogen atom, to which they are attached, form a heterocyclic ring Het5, in which
Het5 is pyrrolidinyl or morpholinyl,
   or,
   in a second aspect (aspect 2) according to the present invention,
R7 is —NH—N(R18)R19, in which
R18 is hydrogen,
R19 is —C(O)R20, or R21-substituted phenyl, in which
R20 is pyridinyl, morpholin-4-yl, 1N—(R203)₄N—(R204)-piperazinyl, or Aryl-methyl, in which
R203 is methyl,
R204 is methyl,
Aryl is 3,4-dimethoxyphenyl,
R21 is aminosulphonyl,
   or R18 and R19 together and with inclusion of the nitrogen atom, to which they are attached, form a heterocyclic ring Het7, in which
Het7 is morpholinyl or 4N—(R181)-piperazinyl, in which
R181 is methyl,
whereby —C(O)R7 is bonded in the meta or para position with respect to the binding position, in which the phenyl ring is attached to the phenanthridine ring system,
   and the salts, the N-oxides and the salts of the N-oxides of these compounds.

Other compounds of formula I in further more particular worthy to be mentioned are those in which
R1 is methoxy,
R2 is ethoxy, 2,2-difluoroethoxy or difluoromethoxy,
R3, R31, R4, R5, R51 and R6 are hydrogen,
R7 is —N(R8)R9, in which
   either
R8 is hydrogen, and
R9 is cyclopropyl or cyclobutyl,
   or
R8 is isopropyl, and
R9 is isopropyl,
whereby —C(O)R7 is bonded in the meta or para position with respect to the binding position, in which the phenyl ring is attached to the phenanthridine ring system,
   and the salts, the N-oxides and the salts of the N-oxides of these compounds.

Other compounds of formula I in further more particular worthy to be mentioned are those in which
R1 is methoxy,
R2 is ethoxy or difluoromethoxy,
R3, R31, R4, R5, R51 and R6 are hydrogen,
R7 is —N(R8)R9, in which
   either
R8 is hydrogen, and
R9 is cyclopropyl or cyclobutyl,
   or
R8 is isopropyl, and
R9 is isopropyl,
whereby —C(O)R7 is bonded in the meta or para position with respect to the binding position, in which the phenyl ring is attached to the phenanthridine ring system,
   and the salts, the N-oxides and the salts of the N-oxides of these compounds.

Other compounds of formula I in further more particular worthy to be mentioned are those in which
R1 is methoxy,
R2 is 2,2-difluoroethoxy,
R3, R31, R4, R5, R51 and R6 are hydrogen,
R7 is —N(R8)R9, in which
   either
R8 is hydrogen, and
R9 is cyclopropyl or cyclobutyl,
   or
R8 is isopropyl, and
R9 is isopropyl,
whereby —C(O)R7 is bonded in the meta or para position with respect to the binding position, in which the phenyl ring is attached to the phenanthridine ring system,
   and the salts, the N-oxides and the salts of the N-oxides of these compounds.

A special interest in the compounds according to this invention relates to those compounds which are included—within the meaning of this invention—by one or, when possible, by more of the following embodiments:

A special embodiment of the compounds of the present invention include those compounds of formula I in which R1 and R2 are independently 1-2C-alkoxy, 2,2-difluoroethoxy, or completely or predominantly fluorine-substituted 1-2C-alkoxy.

Another special embodiment of the compounds of the present invention include those compounds of formula I in which R1 and R2 are independently 1-2C-alkoxy, 2,2-difluoroethoxy, or completely or predominantly fluorine-substituted 1-2C-alkoxy, and R3, R31, R4, R5 and R51 are all hydrogen.

Another special embodiment of the compounds of the present invention include those compounds of formula I in which R1 and R2 are independently 1-2C-alkoxy, 2,2-difluoroethoxy, or completely or predominantly fluorine-substituted 1-2C-alkoxy, and R3, R31, R4, R5, R51 and R6 are all hydrogen.

Another special embodiment of the compounds of the present invention include those compounds of formula I in which one of R1 and R2 is methoxy, and the other is methoxy, ethoxy, difluoromethoxy or 2,2-difluoroethoxy, and R3, R31, R4, R5, R51 and R6 are all hydrogen.

Another special embodiment of the compounds of the present invention include those compounds of formula I in which R1 is ethoxy or, particularly, methoxy, and R2 is methoxy, or, particularly, ethoxy, difluoromethoxy or 2,2-difluoroethoxy, and R3, R31, R4, R5, R51 and R6 are all hydrogen.

Another special embodiment of the compounds of the present invention include those compounds of formula I in which R1 is methoxy, and R2 is methoxy, ethoxy, difluoromethoxy or 2,2-difluoroethoxy, and R3, R31, R4, R5, R51 and R6 are all hydrogen.

Another special embodiment of the compounds of the present invention include those compounds of formula I in which R1 is methoxy, and R2 is ethoxy, difluoromethoxy or 2,2-difluoroethoxy, and R3, R31, R4, R5, R51 and R6 are all hydrogen.

Another special embodiment of the compounds of the present invention include those compounds of formula I in which one of R1 and R2 is 2,2-difluoroethoxy, and R3, R31, R4, R5, R51 and R6 are all hydrogen.

Another special embodiment of the compounds of the present invention include those compounds of formula I in which R1 is ethoxy or, particularly, methoxy, and R2 is 2,2-difluoroethoxy, and R3, R31, R4, R5, R51 and R6 are all hydrogen.

Another special embodiment of the compounds of the present invention include those compounds of formula I in which R1 is methoxy, and R2 is 2,2-difluoroethoxy, and R3, R31, R4, R5, R51 and R6 are all hydrogen.

Another special embodiment of the compounds of the present invention include those compounds of formula I in which R1 is methoxy, and R2 is ethoxy, and R3, R31, R4, R5, R51 and R6 are all hydrogen.

Another special embodiment of the compounds of the present invention include those compounds of formula I in which R1 is methoxy, and R2 is difluoromethoxy, and R3, R31, R4, R5, R51 and R6 are all hydrogen.

Another special embodiment of the compounds of the present invention include those compounds of formula I in which R6 is hydrogen.

Another special embodiment of the compounds of the present invention include those compounds of formula I according to aspect 1.

Another special embodiment of the compounds of the present invention include those compounds of formula I according to aspect 2.

Another special embodiment of the compounds of the present invention include those compounds of formula I according to aspect 1 in which R9 is pyridinyl substituted by R91 and R92.

Another special embodiment of the compounds of the present invention include those compounds of formula I according to aspect 1 in which R8 is hydrogen or 1-4C-alkyl, and R9 is 1-4C-alkyl, cyclopropyl or cyclobutyl.

Another special embodiment of the compounds of the present invention include those compounds of formula I according to aspect 1, in which R8 is isopropyl and R9 is isopropyl.

Another special embodiment of the compounds of the present invention include those compounds of formula I according to aspect 1 in which R8 is hydrogen and R9 is cyclopropyl or cyclobutyl.

Another special embodiment of the compounds of the present invention include those compounds of formula I according to aspect 1, in which R8 is isopropyl and R9 is isopropyl.

The compounds of the formula I are chiral compounds having chiral centers at least in positions 4a and 10b and, depending on the meaning of the substituents R3, R31, R4, R5 and R51, further chiral centers in the positions 1, 2, 3 and 4.

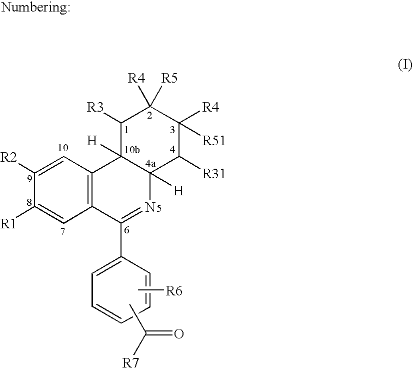

The invention therefore comprises all conceivable stereoisomers in pure form as well as in any mixing ratio, and the salts thereof.

Preferred compounds of the formula I are those in which the hydrogen atoms in positions 4a and 10b are in the ds position relative to one another. The pure cis diastereomers, the pure cis enantiomers and their mixtures in any mixing ratio and including the racemates are more preferred in this context.

Particularly preferred in this connection are those compounds of the formula I which have, with respect to the positions 4a and 10b, the same configuration as shown in the formula I*:

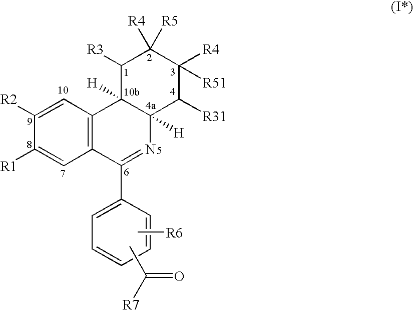

If, for example in compounds of the formula I* R3, R31, R4, R5 and R51 have the meaning hydrogen, then the configuration—according the rules of Cahn, Ingold and Prelog—is R in the position 4a and R in the position 10b.

The enantiomers can be separated in a manner known per se (for example by preparation and separation of appropriate diastereoisomeric compounds). For example, an enantiomer separation can be carried out at the stage of the starting compounds of the formula VII in which R1, R2, R3, R31, R4, R5 and R51 have the meanings indicated above.

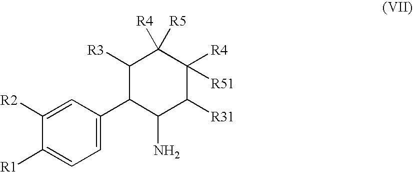

Separation of the enantiomers can be carried out, for example, by means of salt formation of the racemic compounds of the formula VII with optically active acids, preferably carboxylic acids, subsequent resolution of the salts and release of the desired compound from the salt. Examples of optically active carboxylic acids which may be mentioned in this connection are the enantiomeric forms of mandelic acid, tartaric acid, O,O′-dibenzoyltartaric acid, camphoric acid, quinic acid, glutamic acid, malic acid, camphorsulfonic acid, 3-bromocamphorsulfonic acid, α-methoxyphenylacetic acid, α-methoxy-α-trifluoromethylphenylacetic acid and 2-phenylpropionic acid. Alternatively, enantiomerically pure starting compounds of the formula VII can be prepared via asymmetric syntheses. Enantiomerically pure starting compounds as well as enantiomerically pure compounds of the formula I can be also obtained by chromatographic separation on chiral separating columns; by derivatization with chiral auxiliary reagents, subsequent diastereomer separation and removal of the chiral auxiliary group; or by (fractional) crystallization from a suitable solvent.

The compounds according to the invention can be prepared, for example, as shown in the reaction schemes below and according to the following specified reaction steps, or, particularly, in a manner as described by way of example in the following examples, or analogously or similarly thereto according to preparation procedures or synthesis strategies known to the person skilled in the art.

Compounds of formula I, in which R1, R2, R3, R31, R4, R5, R51, R6 and R7 have the meanings mentioned above, can be obtained as outlined in reaction scheme 1 and as described as follows starting with compounds of formula IV, in which C(O)OR stands for a suitable ester group such as an alkyl ester (preferably a methyl ester group).

On the one hand, compounds of formula I may be obtained from the compounds of formula IV by direct reaction with compounds of formula R7-H, in which R7 has the meanings given above.

On the other hand the compounds of formula IV can be first saponified to give the benzoic acid derivatives of formula II, which can be amidified with compounds of formula R7-H in a manner known to the skilled person.

Thus, e.g. benzoic acid derivatives of formula III can be activated prior to the amide bond forming reaction with compounds of formula R7-H, for example by forming an acid halide or acid anhydride, (compounds of formula II, in which Y is a suitable leaving group), or by using coupling agents known to the person skilled in the art, such as, for example, N,N′-dicyclohexylcarbodiimide, N′-(3-dimethylaminopropyl)-N-ethylcarbodiimide hydrochloride (EDCl) or 2-(1H-benzotriazole-1-yl)-1,3,3-tetramethyluronium hexafluorophosphate (HBTU).

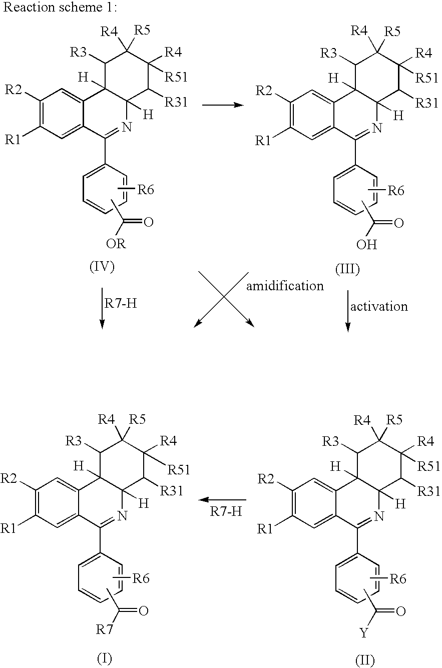

Optionally, compounds of the formula I can be converted into their salts, or, optionally, salts of the compounds of the formula I can be converted into the free compounds.

In addition, the compounds of the formula I can be converted, optionally, into their N-oxides, for example with the aid of hydrogen peroxide in methanol or with the aid of m-chloroperoxybenzoic acid in dichloromethane. The person skilled in the art is familiar on the basis of his/her expert knowledge with the reaction conditions which are specifically necessary for carrying out the N-oxidation.

Compounds of the formula IV, in which R1, R2, R3, R31, R4, R5, R51, R6 and COOR have the meanings indicated above, can be prepared according to those procedures given by way of example in the following examples. For greater detail, a suitable synthesis route for compounds of the formula IV is outlined in reaction scheme 2 below. In the first step of said reaction scheme 2 compounds of the formula VII, in which R1, R2, R3, R31, R4, R5 and R51 have the meanings given above, are reacted with compounds of the formula VI, in which R6 and COOR have the meanings given above and X represents a suitable leaving group, preferably a chlorine atom, to give corresponding compounds of the formula V.

Alternatively, compounds of the formula V, in which R1, R2, R3, R31, R4, R5, R51, R6 and COOR have the meanings given above, can also be prepared, for example, from compounds of the formula VII, in which R1, R2, R3, R31, R4, R5 and R51 have the abovementioned meanings, and compounds of the formula VI, in which R6 and COOR have the abovementioned meanings and X is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O-(benzotriazol-1-yl)-N,N,N′,N′-tetramethyluronium tetrafluoroborate or O-(benzotriazol-1-yl)-N, N,N′,N′-tetramethyl-uronium-hexafluorophosphate] and N,N′-carbonyldiimidazole. In the scope of this invention preferred amide bond linking reagents are uronium salts and, particularly, carbodiimides, preferably, 1-ethyl-3-(3-dimethylaminopropyl)carbodiimide hydrochloride.

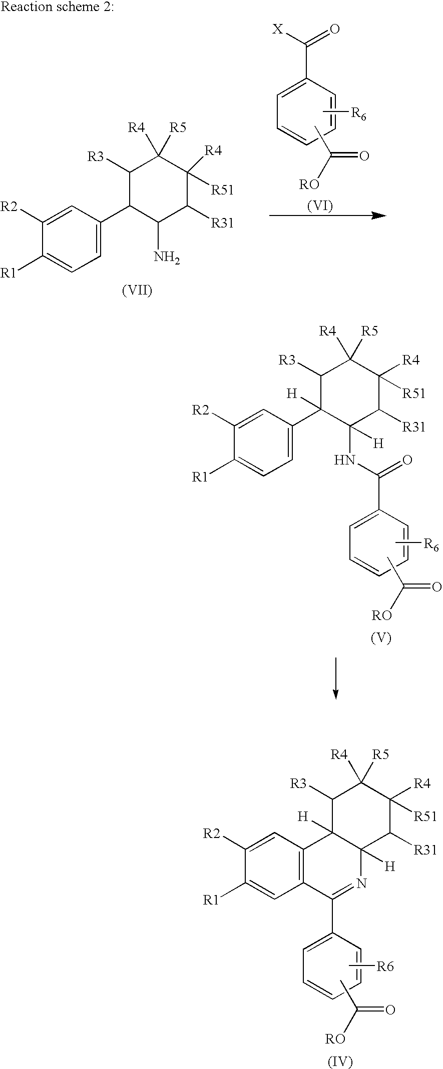

Compounds of the formula VI are either known or can be prepared in according to known procedures.

As shown in the next step within reaction scheme 1, compounds of the formula IV, in which R1, R2, R3, R31, R4, R5, R51, R6 and COOR have the meanings indicated above, can be obtained by cyclocondensation of corresponding compounds of the formula V. Said cyclocondensation reaction is carried out in a manner habitual per se to the person skilled in the art or as described by way of example in the following examples, according to Bischler-Napieralski (e.g. as described in J. Chem. Soc., 1956, 4280-4282) in the presence of a suitable condensing agent, such as, for example, polyphosphoric acid, phosphorus pentachloride, phosphorus pentoxide or phosphorus oxychloride, in a suitable inert solvent, e.g. in a chlorinated hydrocarbon such as chloroform, or in a cyclic hydrocarbon such as toluene or xylene, or another inert solvent such as acetonitrile, or without further solvent using an excess of condensing agent, at reduced temperature, or at room temperature, or at elevated temperature or at the boiling temperature of the solvent or condensing agent used.

If necessary, said cyclocondensation reaction can be carried out in the presence of one or more suitable Lewis Acids such as, for example, suitable metal halogenides (e.g. chlorides) or sulphonates (e.g. triflates), including rare earth metal salts, such as e.g. anhydrous aluminum trichloride, aluminum tribromide, zinc chloride, boron trifluoride ethereate, titanium tetrachloride or, in particular, tin tetrachloride, and the like.

The preparation of pure enantiomers of starting compounds of the formula VII can be carried out as described, for example, in the international application WO00/42020 or in a manner according to the following examples.

It is also known to the person skilled in the art that, if a plurality of reactive centers are present in a starting material or intermediate, it may be necessary to temporarily block one or more reactive centers with protective groups so that a reaction takes place only at the desired reactive center. A detailed description of how to use a large number of proven protective groups can be found, for example, in T. W. Greene, Protective Groups in Organic Synthesis, John Wiley & Sons, 1991 or 1999 (3^{rd} edition), or in “Protecting Groups (Thieme Foundations Organic Chemistry Series N Group)” by P. Kocienski (Thieme Medical Publishers, 2000).

The substances according to the invention are isolated and purified in a manner known per se, for example by distilling off the solvent under reduced pressure and recrystallizing the residue obtained from a suitable solvent or subjecting it to one of the customary purification methods, such as, for example, column chromatography on a suitable support material.

Salts are obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low-molecular-weight aliphatic alcohol, such as methanol, ethanol or isopropanol) which contains the desired acid or base, or to which the desired acid or base is then added. The salts are obtained by filtering, reprecipitating, precipitating with a nonsolvent for the addition salt or by evaporating the solvent. Salts obtained can be converted into the free compounds, which can in turn be converted into salts, by alkalization or by acidification. In this manner, pharmacologically unacceptable salts can be converted into pharmacologically acceptable salts.

Suitably, the conversions mentioned in this invention can be carried out analogously or similarly to methods which are familiar per se to the person skilled in the art.

The person skilled in the art knows on the basis of his/her knowledge and on the basis of those synthesis routes, which are shown and described within the description of this invention, how to find other possible synthesis routes for compounds of the formula I. All these other possible synthesis routes are also part of this invention.

The present invention also relates to intermediates, including their salts, methods and processes useful in synthesizing compounds according to this invention.

Having described the invention in detail, the scope of the present invention is not limited only to those described characteristics or embodiments. As will be apparent to persons skilled in the art, modifications, analogies, variations, derivations, homologisations and adaptations to the described invention can be made on the base of art-known knowledge and/or, particularly, on the base of the disclosure (e.g. the explicite, implicite or inherent disclosure) of the present invention without departing from the spirit and scope of this invention as defined by the scope of the appended claims.

The following examples serve to illustrate the invention further without restricting it. Likewise, further compounds of the formula I, whose preparation is not explicitly described, can be prepared in an analogous or similar manner or in a manner familiar per se to the person skilled in the art using customary process techniques.

Any or all of the compounds of formula I which are mentioned in the following examples as final compounds as well as their salts, N-oxides and salts of the N-oxides are a preferred subject of the present invention.

In the examples, m.p. stands for melting point, h for hour(s), min for minutes, R_{f} for rentention factor in thin layer chromatography, s.p. for sintering point, EF for empirical formula, MW for molecular weight, MS for mass spectrum, M for molecular ion, fnd. for found, calc. for calculated, other abbreviations have their meanings customary per se to the skilled person.

### EXAMPLES

### Final Compounds

Starting from the appropriate starting compound, the following final compounds can be obtained analogously or similarly as described for Example 8.
1. ({1-[4-((4aR,10bR)-8,9-Dimethoxy-1,2,3,4,4a,10b-hexahydro-phenanthridin-6-yl)-phenyl]-methanoyl}-amino)-acetic acid methyl ester

C₂₅H₂₈N₂O₅

Calc.: 436.51 Found (MH⁺): 437.2
2. 4-((4aR,10bR)-8,9-Dimethoxy-1,2,3,4,4a,10b-hexahydro-phenanthridin-6-yl)-N-(2-methoxyethyl)-N-methyl-benzamide

C₂₆H₃₂N₂O₄

Calc.: 436.56 Found (MH⁺): 437.2
3. 4-((4aR,10bR)-8,9-Dimethoxy-1,2,3,4,4a,10b-hexahydro-phenanthridin-6-yl)-N-(1-methoxymethyl-propyl)-benzamide

C₂₇H₃₄N₂O₄

Calc.: 450.58 Found (MH⁺): 451.2
4. 4-((4aR,10bR)-8,9-Dimethoxy-1,2,3,4,4a,10b-hexahydro-phenanthridin-6-yl)-N-(2-methoxyethyl)-benzamide

C₂₅H₃₀N₂O₄

Calc.: 422.53 Found (MH⁺): 423.2
5. N-(2,2-Diethoxy-ethyl)-4-((4aR,10bR)-8,9-dimethoxy-1,2,3,4,4a,10b-hexahydrophenanthridin-6-yl)-benzamide

C₂₈H₃₆N₂O₅

Calc.: 480.61 Found (MH⁺): 481.1
6. 4-((4aR,10bR)-8,9-Dimethoxy-1,2,3,4,4a,10b-hexahydro-phenanthridin-6-yl)-N-(3-hydroxypropyl)-benzamide

C₂₅H₃₀N₂O₄

Calc.: 422.53 Found (MH⁺): 423.2
7. 1-[4-((4aR,10bR)-Dimethoxy-1,2,3,4,4a,10b-hexahydro-phenanthridin-6-yl)-phenyl]-1-[4-(2-dimethylamino-ethyl)-piperazin-1-yl]-methanone

C₃₀H₄₀N₄O₃

Calc.: 504.68 Found (MH⁺): 505.4
8. (4-{1-[4-((4aR,10bR)-8,9-Dimethoxy-1,2,3,4,4a,10b-hexahydro-phenanthridin-6-yl)-phenyl]-methanoyl}-piperazin-1-yl)-dimethylamino-ethanone C₃₀H₃₈N₄O₄

402 mg (1.01 mmol) of the compound A1, 198 mg (1.12 mmol) of piperazino-acetic acid-N,N-dimethylamide, 171 mg (1.1 mmol) of 1-ethyl-3-(3-dimethylaminopropyl)carbodiimide hydrochloride are dissolved in 6 ml of dichloromethane and 10 mg of p-dimethylaminopyridine are added. After stirring for 4 h the solvent is removed and the residue purified by means of flash chromatography to yield 280 mg (54%) of the title compound.

Calc.: 518.66 Found (MH⁺): 519.4
9. 1-(4-{1-[4-((4aR,10bR)-8,9-Dimethoxy-1,2,3,4,4a,10b-hexahydro-phenanthridin-6-yl)phenyl]-methanoyl}-piperazin-1-yl)-2-morpholin-4-yl-ethanone

C₃₂H₄₀N₄O₅

Calc.: 560.7 Found (MH⁺): 561.5
10. 4-((4aR,10bR)-8,9-Dimethoxy-1,2,3,4,4a,10b-hexahydro-phenanthridin-6-yl)-N-(4-methyl-piperazin-1-yl)-benzamide

C₂₇H₃₄N₄O₃

Calc.: 462.60 Found (MH⁺): 463.4
11. 4-((4aR,10bR)-8,9-Dimethoxy-1,2,3,4,4a,10b-hexahydro-phenanthridin-6-yl)-N-morpholin-4-yl-benzamide

C₂₆H₃, N₃O₄

Calc.: 449.55 Found (MH⁺): 450.3
12. 4-((4aR,10bR)-8,9-Dimethoxy-1,2,3,4,4a,10b-hexahydro-phenanthridin-6-yl)-N-(2-morpholin-4-yl-ethyl)-benzamide

C₂₈H₃₅N₃O₄

Calc.: 477.61 Found (MH⁺): 478.4
13. Nicotinic acid N′-{1-[4-((4aR,10bR)-8,9-dimethoxy-1,2,3,4,4a,10b-hexahydrophenanthridin-6-yl)-phenyl]-methanoyl}-hydrazide

C₂₈H₂₈N₄O₄

Calc.: 484.56 Found (MH⁺): 485.3
14. 4-((4aR,10bR)-8,9-Dimethoxy-1,2,3,4,4a,10b-hexahydro-phenanthridin-6-yl)-benzoic acid N′-[2-(3,4-dimethoxy-phenyl)-ethanoyl]-hydrazide

C₃₂H₃₅N₃O₆

Calc.: 557.65 Found (MH⁺): 558.4
15. Morpholine-4-carboxylic acid N′-{1-[4-((4aR,10bR)-8,9-dimethoxy-1,2,3,4,4a,10b-hexahydro-phenanthridin-6-yl)-phenyl]-methanoyl}-hydrazide

C₂₇H₃₂N₄O₅

Calc.: 492.58 Found (MH⁺): 493.2
16. 4-(N′-{1-[4-((4aR,10bR)-8,9-Dimethoxy-1,2,3,4,4a,10b-hexahydro-phenanthridin-6-yl)phenyl]-methanoyl}-hydrazino)-benzenesulfonamide

C₂₈H₃₀N₄O₅S

Calc.: 534.64 Found (MH⁺): 535.5
17. Isonicotinic acid N′-{1-[4-((4aR,10bR)-8,9-dimethoxy-1,2,3,4,4a,10b-hexahydrophenanthridin-6-yl)-phenyl]-methanoyl}-hydrazide

C₂₈H₂₈N₄O₄

Calc.: 484.56 Found (MH⁺): 485.3
18. 1,4-Dimethyl-piperazine-2-carboxylic acid N′-{1-[4-((4aR,10bR)-8,9-dimethoxy-1,2,3,4,4a,10b-hexahydro-phenanthridin-6-yl)-phenyl]-methanoyl}-hydrazide

C₂₉H₃₇N₅O₄

Calc.: 519.65 Found (MH⁺): 520.4
19. ({1-[4-((4aR,10bR)-8,9-Dimethoxy-1,2,3,4,4a,10b-hexahydro-phenanthridin-6-yl)-phenyl]-methanoyl}-methyl-amino)-acetic acid methyl ester

C₂₆H₃₀N₂O₅

Calc.: 450.54 Found (MH⁺): 451.4
20. 4-((4aR,10bR)-8,9-Dimethoxy-1,2,3,4,4a,10b-hexahydro-phenanthridin-6-yl)-N-(3-morpholin-4-yl-propyl)-benzamide

C₂₉H₃₇N₃O₄

Calc.: 494.64 Found (MH⁺): 492.3
21. 1-{4-[(4aR,10bR)-9-(1,1-Difluoro-methoxy)-8-methoxy-1,2,3,4,4a,10b-hexahydrophenanthridin-6-yl]-phenyl}-1-[4-(2-morpholin-4-yl-ethyl)-piperazin-1-yl]-methanone

C₃₂H₄₀F₂N₄O₄

Calc.: 582.70 Found (MH⁺): 583.4
22. 2-[4-(1-{4-[(4aR,10bR)-9-(1,1-Difluoro-methoxy)-8-methoxy-1,2,3,4,4a,10b-hexahydrophenanthridin-6-yl]-phenyl}-methanoyl)-piperazin-1-yl]-N,N-dimethyl-acetamide

C₃₀H₃₆F₂N₄O₄

Calc.: 554.64 Found (MH⁺): 555.3
23. 4-[(4aR,10bR)-9-(1,1-Difluoro-methoxy)-8-methoxy-1,2,3,4,4a,10b-hexahydrophenanthridin-6-yl]-N-(3-morpholin yl-propyl)-benzamide

C₂₉H₃₅F₂N₃O₄

Calc.: 527.62 Found (MH⁺): 528.3
24. [(1-{4-[(4aR,10bR)-9-(1,1-Difluoro-methoxy)-8-methoxy-1,2,3,4,4a,10b-hexahydrophenanthridin-6-yl]-phenyl)-methanoyl}-amino]-acetic acid methyl ester

C₂₅H₂₆F₂N₂O₅

Calc.: 472.49 Found (MH⁺): 473.3
25. [(1-{4-[(4aR,10bR)-9-(1,1-Difluoro-methoxy)-8-methoxy-1,2,3,4,4a,10b-hexahydrophenanthridin-6-yl]-phenyl}-methanoyl)-methyl-amino]-acetic acid methyl ester

C₂₆H₂₈F₂N₂O₅

Calc.: 486.52 Found (MH⁺): 487.3
26. 4-[(4aR,10bR)-9-(1,1-Difluoro-methoxy)-8-methoxy-1,2,3,4,4a,10b-hexahydrophenanthridin-6-yl]-N-(2-pyridin-3-yl-ethyl)-benzamide

C₂₉H₂₉F₂N₃O₃

Calc.: 505.57 Found (MH⁺): 506.4
27. 1-{4-[(4aR,10bR)-9-(1,1-Difluoro-methoxy)-8-methoxy-1,2,3,4,4a,10b-hexahydrophenanthridin-6-yl]-phenyl}-1-(4-pyridin-4-yl-piperazin-1-yl)-methanone

C₃₁H₃₂F₂N₄O₃

Calc.: 546.62 Found (MH⁺): 547.3
28. 4-[(4aR,10bR)-9-(1,1-Difluoro-methoxy)-8-methoxy-1,2,3,4,4a,10b-hexahydrophenanthridin-6-yl]-N-(2-methoxy-ethyl)-N-methyl-benzamide

C₂₆H₃₀F₂N₂O₄

Calc.: 472.54 Found (MH⁺): 473.4
29. 4-((4aR,10bR)-8,9-Dimethoxy-1,2,3,4,4a,10b-hexahydro-phenanthridin-6-yl)-N-ethyl-N-(2-methoxy-ethyl)-benzamide

C₂₇H₃₄N₂O₄

Calc.: 450.58 Found (MH⁺): 451.4
30. 4-((4aR,10bR)-8,9-Dimethoxy-1,2,3,4,4a,10b-hexahydro-phenanthridin-6-yl)-N-ethyl-N-(2-methoxy-ethyl)-benzamide

C₂₇H₃₄N₂O₄

Calc. 450.58 Found (MH⁺) 451.4
31. 1-[4-((4aR,10bR)-8,9-Dimethoxy-1,2,3,4,4a,10b-hexahydro-phenanthridin-6-yl)-phenyl]-1-[4-(2-morpholin-4-yl-ethyl)-piperazin-1-yl]-methanone

C₃₂H₄₂N₄O₄

Calc. 546.72 Found (MH⁺) 547.5
32. 4-((4aR,10bR)-8,9-Dimethoxy-1,2,3,4,4a,10b-hexahydro-phenanthridin-6-yl)-N-[2-(4-methyl-piperazin-1-yl)-ethyl]-benzamide

C₂₉H₃₈N₄O₃

Calc. 490.65 Found (MH⁺) 491.3
33. 2-(4-{1-[4-((4aR,10bR)-8,9-Dimethoxy-1,2,3,4,4a,10b-hexahydro-phenanthridin-6-yl)phenyl]-methanoyl}-piperazin-1-yl)-N,N-dimethyl-acetamide

C₃₀H₄₀Cl₂ N₄O₄

Calc. 591.58 (Dihydrochloride); 518.66 (free base) Found (MH⁺) 519.4

Starting from the appropriate starting compounds described below or obtainable analogously or similarly as described herein, and the appropriate art-known amines further compounds can be obtained analogously as described for Example 8.

### Starting Compounds

A1. 4-[(4aR,10bR)-8,9-Dimethoxy-1,2,3,4,4a,10b-hexahydrophenanthridin-6-yl)]-benzoic acid hydrochloride

17 g 4-[(4aR,10bR)-8,9-Dimethoxy-1,2,3,4,4a,10b-hexahydro-phenanthridin-6-yl]-benzoic acid methyl ester (compound A3) are dissolved in 100 ml water and 50 ml conc. hydrochloric acid and stirred at 80° C. for 3 h. The solvent is removed under reduced pressure and the residue is crystallized from methyl ethyl ketone and methanol. After filtering and drying 12.8 g of the title compound are obtained of melting point 228° C. (decomp.).
A2. 3-[(4aR,10bR)-4,9-Dimethoxy-1,2,3,4,4a,10b-hexahydro-phenanthridin-6-yl]-benzoic acid hydrochloride

The title compound can be prepared analogously as described for starting compound A1.

MS: calc.: C₂₂H₂₄Cl N O_{4[}365.43+(HCl) 36.46] fnd.: [M+1] 366.2
A3. 4-[(4aR,1bR)-8,9-Dimethoxy-1,2,3,4,4a,10b-hexahydro-phenanthridin-6-yl]-benzoic acid methyl ester

42.7 g N-[(1R,2R)-2-(3,4-Dimethoxy-phenyl)-cyclohexyl]-terephthalamic acid methyl ester (compound A5) and 25 ml phosphorus oxychloride are dissolved in 500 ml acetonitril and stirred overnight at 80° C. The solvent is evaporated under reduced pressure, the residue is dissolved in ethyl acetate and extracted with sodium bicarbonate solution. The organic layer is dried over sodium sulfate and concentrated. The crude product is purified by chromatography on silica gel using a mixture of petroleum ether/ethyl acetate/triethylamin in the ratio 6/3/1 to furnish 37.7 g of the title compound with a optical rotation of [α]_{D}²⁰=82 (c=0.2, Ethanol).
A4. 3-((4aR,10bR)-8,9-Dimethoxy-1,2,3,4,4a,10b-hexahydro-phenanthridin-6-yl)-benzoic acid methyl ester

The title compound can be analogously prepared as described for starting compound A3.

M.p. 110-111° C.
A5. N-[(1R,2R)-2-(3,4-Dimethoxy-phenyl)-cyclohexyl]-terephthalamic acid methyl ester

27.2 g 1,2-Dimethoxy-4-[1R-(2R-aminocyclohexyl)]benzene (compound B1) are dissolved in 300 ml methylene chloride and 50 ml triethylamine. A solution of 27.2 g 4-chlorocarbonyl-benzoic acid methyl ester in 300 ml methylene chloride is added dropwise at room temperature and the mixture is stirred overnight. The solution is extracted with water, 1 M hydrochloric acid, sodium bicarbonate solution and water. The organic layer is dried over sodium sulfate and the solvent is evaporated to yield 43.4 g of the title compound with melting point 154-156° C.
A6. N-[(1R,2R)-2-(3,4-Dimethoxy-phenyl)-cyclohexyl]-isophthalamic acid methyl ester

The title compound can be prepared analogously as described for starting compound A5.

M.p. 108-109° C.
B1. (1R,2R)-2-(3,4-Dimethoxyphenyl)-cyclohexylamine

12.0 g of a racemic mixture of (1R,2R)-2-(3,4-dimethoxyphenyl)-cyclohexylamine and (1S,2S)-2-(3,4-dimethoxyphenyl)-cyclohexylamine and 6.2 g of (−)-mandelic acid are dissolved in 420 ml of dioxane and 60 ml of tetrahydrofuran and the solution is stirred overnight at RT. The solid is filtered off with suction, dried, treated with 100 ml of saturated sodium hydrogencarbonate solution and extracted with ethyl acetate. The organic phase is dried using sodium sulfate and concentrated under reduced pressure. 4.8 g of the title compound are obtained of m.p.: 80-81.5° C.

Specific rotation: [α]_{D}²⁰=−58.5° C. (c=1, ethanol).

Starting from the appropriate starting compound, the following compounds B2 to B4 and further relevant starting compounds can be obtained analogously or similarly as described for compound B1.
B2. (1R,2R)-2-(3-Ethoxy-4-methoxy-phenyl)-cyclohexylamine
B3. (1R,2R)-2-[3-(2,2-Difluoroethoxy)methoxy-phenyl]cyclohexylamine
B4. (1R,2R)-2-[3-Difluoromethoxy methoxy-phenyl]-cyclohexylamine
B5. (1R,2R)-2-[4-Difluoromethoxy-3-methoxy-phenyl]-cyclohexylamine
C1. Racemic mixture of (1R,2R)-2-(3,4-dimethoxyphenyl)-cyclohexylamine and (1S,2S)-2-(3,4-dimethoxyphenyl)-cyclohexylamine

125 g of a racemic mixture of 1,2-dimethoxy-4-((1R,2R)-2-nitrocyclohexyl)benzene and 1,2-dimethoxy-4-((1S,2S)-2-nitrocyclohexyl)benzene and 120 g of zinc powder or granules are suspended in 1300 ml of ethanol. 220 ml of acetic acid are added dropwise at boiling heat. The precipitate is filtered off with suction and washed with ethanol, and the filtrate is concentrated under reduced pressure. The residue is taken up in hydrochloric acid and extracted with toluene. The aqueous phase is rendered alkaline using 50% strength sodium hydroxide solution, the precipitate is filtered off with suction and the filtrate is extracted with toluene. The organic phase is dried using sodium sulfate and concentrated. 98 g of the title compound are obtained as a crystallizing oil.

### Alternatively:

8.5 g of a racemic mixture of 1,2-dimethoxy-4-((1R,2R)-2-nitrocyclohexyl)benzene and 1,2-dimethoxy-4-((1S,2S)-2-nitrocyclohexyl)benzene are dissolved in 400 ml of methanol and treated at RT with 7 ml of hydrazine hydrate and 2.5 g of Raney nickel in portions in the course of 8 h. After stirring overnight at RT, the reaction mixture is filtered, the filtrate is concentrated and the residue is chromatographed on silica gel using a mixture of toluene/ethyl acetate/triethylamine=41210.5. The title compound is obtained as an oil.
C2. (1RS,2RS)-2-[3-(2,2-Difluoroethoxy)-4-methoxy-phenyl]-cyclohexylamine

13.2 g (42.1 mmol) of compound D2 and 13.775 g (210 mmol) of zinc are suspended in 100 ml of ethanol and heated to reflux. 18.5 ml of acetic acid dissolved in 30 ml of ethanol are added dropwise over 2 h. The mixture is stirred for another 2 h. The solids are filtered off and the remaining solution is evaporated and then treated with 200 ml of chloroform and 100 ml of 6 N NaOH. The organic layer is separated and the solvent removed to give 13.9 g of an oily residue. The product is treated with 100 ml of chloroform and 100 ml of 1 N HCl. The organic layer is separated and discarded, the pH of the water layer is brought to alkaline adding 6 N NaOH and then extracted with chloroform. After removing the solvent 6.5 g of the title compound (54%) are obtained as a yellowish oil.
D1. Racemic mixture of 1,2-dimethoxy-4-((1R,2R)-2-nitrocyclohexyl)benzene and 1,2-dimethoxy-4-((1S,2S)-2-nitrocyclohexyl)benzene

8.4 g of a racemic mixture of 1,2-dimethoxy-4-((1R,2R)-2-nitrocyclohex-4-enyl)benzene and 1,2-dimethoxy-4-((1S,2S)-2-nitrocyclohex-4-enyl)benzene are dissolved in 450 ml of methanol, treated with 2 ml of conc. hydrochloric acid and hydrogenated after addition of 500 mg of 10% strength Pd/C. The reaction mixture is filtered and the filtrate is concentrated. M.p.: 84-86.5° C.
D2. 2-(2,2-Difluoro-ethoxy)-1-methoxy-4-((1RS,2RS)-2-nitro-cyclohexyl)-benzene

15.3 g (48.8 mmol) of compound E2 are dissolved in toluene (100 ml) and 100 mg catalyst (Pd on C) are added. The mixture is stirred under a hydrogen atmosphere (1 bar) until no more hydrogen is consumed. The solids are filtered off (celite) and the solvent is removed to yield 14.2 g of a colorless oil which is used for the following step without further purification
E1. Racemic mixture of 1,2-dimethoxy-4-((1R,2R)-2-nitrocyclohex-4-enyl)benzene and 1,2-dimethoxy-4-((1S,2S)-2-nitrocyclohex-4-enyl)benzene

10.0 g of a racemic mixture of 1,2-dimethoxy-4-((1R,2S)-2-nitrocyclohex-4-enyl)benzene and 1,2-dimethoxy-4-((1S,2R)-2-nitrocyclohex-4-enyl)benzene and 20.0 g of potassium hydroxide are dissolved in 150 ml of ethanol and 35 ml of dimethylformamide. A solution of 17.5 ml of conc. sulfuric acid in 60 ml of ethanol is then added dropwise such that the internal temperature does not exceed 4° C. After stirring for 1 h, the mixture is added to 1 l of ice water, the precipitate is filtered off with suction, washed with water and dried, and the crude product is recrystallized from ethanol. 8.6 g of the title compound of m.p. 82.5-84° C. are obtained.
E2. 2-(2,2-Difluoro-ethoxy)-1-methoxy-4-((1RS,6RS)-6-nitro-cyclohex-3-enyl)-benzene

41.00 g (130.9 mmol) of compound F2 are dissolved in 650 ml of dimethylformamide. 53 ml of sodium methanolate (30% in methanol) are added dropwise. After cooling to −5° C. a mixture of 62.5 ml of phosphoric acid (85%) and 250 ml of methanol is added. The mixture is poured into water and extracted twice with diethyl ether. The combined organic layers are extracted with water and dried over sodium sulfate. After removing the solvent the remaining oil is purified by means of chromatography on silica. The resulting residue is recrystallized from ethanol to yield 15.3 g (37%) of the title compound.
F1. Racemic mixture of 1,2-dimethoxy-4-((1R,2S)-2-nitrocyclohex-4-enyl)benzene and 1,2-dimethoxy-4-((1S,2R)-2-nitrocyclohex-4-enyl)benzene

50.0 g of 3,4-dimethoxy-nitrostyrene (compound G1) and 1.0 g (9.1 mmol) of hydroquinone are suspended in 200 ml of dry toluene and treated at −70° C. with 55.0 g (1.02 mol) of liquid 1,3-butadiene. The mixture is stirred at 160° C. for 6 days in an autoclave and then cooled. Some of the solvent is removed on a rotary evaporator, and the resulting precipitate is filtered off with suction and recrystallized in ethanol. M.p.: 113.5-115.5° C.
F2. (2,2-Difluoro-ethoxy)-1-methoxy-4-((1RS,6SR)-6-nitro-cyclohex-3-enyl)-benzene

46.00 g (177.5 mmol) of compound G3, 100 mg of hydroquinone and 100 ml of toluene are placed in an autoclave. At −40° C. about 40 g 1,3-butadiene is condensed in the mixture, the autoclave closed and the mixture heated at 160° C. for 16 h. Since starting material is still present another 25 g of 1,3-butadiene are condensed in the autoclave and then heated for another 23 h at 160° C. The solvents are removed and the oily residue recrystallized from ethyl acetate to yield 42.9 g (78%) of the tile compound.
G1. 3,4-Dimethoxy-ω-nitrostyrene

207.0 g of 3,4-dimethoxybenzaldehyde, 100.0 g of ammonium acetate and 125 ml of nitromethane are heated to boiling for 3-4 h in 1.0 l of glacial acetic acid. After cooling in an ice bath, the precipitate is filtered off with suction, rinsed with glacial acetic acid and petroleum ether and dried. M.p.: 140-141° C.

Yield: 179.0 g.

Starting from starting compounds, which are art-known or which can be obtained analogously to art-known compounds or according to art-known procedures (such as e.g. as described in WO 95/01338 or analogously or similarly thereto) the following compounds are obtained according to the procedure as in Example G1:
G2. 3-Ethoxy-4-methoxy-ω-nitrostyrene
G3. 3-(2,2-Difluoro-ethoxy-4)-methoxy-ω-nitrostyrene

The title compound is obtained starting from 3-(2,2-difluoro-ethoxy)-4-methoxy-benzaldehyde (compound H1) according to the procedure as in Example G1.

M.p.: 164-165° C.
G4. 4-(1,1-Difluoro-methoxy)-3-methoxy-ω-nitrostyrene
G5. 3-(1,1-Difluoro-methoxy)-4-methoxy-ω-nitrostyrene
H1. 3-(2,2-Difluoro-ethoxy)-4-methoxy-benzaldehyde

10.04 g of isovanillin and 15.5 g of potassium carbonate are placed in an autoclave. 50 ml of DMF are added as well as 12.44 g of 2-bromo-1,1-difluoroethane. The autoclave is closed and heated at 60° C. for 20 h. Then the solids are filtered off and washed with 120 ml of DMF. About 120 ml of the solvent are distilled off and the residue poured on 200 ml of ice/water, where the product precipitates. After stirring the slurry for 30 minutes the product is filtered off and dried to give 13.69 g of the desired product.

M.p.: 66-68° C.

### Commercial Utility

The compounds according to the invention have useful pharmacological properties which make them industrially utilizable. As selective cyclic nucleotide phosphodiesterase (PDE) inhibitors (specifically of type 4), they are suitable on the one hand as bronchial therapeutics (for the treatment of airway obstructions on account of their dilating action but also on account of their respiratory rate- or respiratory drive-increasing action) and for the removal of erectile dysfunction on account of their vascular dilating action, but on the other hand especially for the treatment of disorders, in particular of an inflammatory nature, e.g. of the airways (asthma prophylaxis), of the skin, of the intestine, of the eyes, of the CNS and of the joints, which are mediated by mediators such as histamine, PAF (platelet-activating factor), arachidonic acid derivatives such as leukotrienes and prostaglandins, cytokines, interleukins, chemokines, alpha-, beta- and gamma-interferon, tumor necrosis factor (TNF) or oxygen free radicals and proteases. In this context, the compounds according to the invention are distinguished by a low toxicity, a good enteral absorption (high bioavailability), a large therapeutic breadth and the absence of significant side effects.

On account of their PDE-inhibiting properties, the compounds according to the invention can be employed in human and veterinary medicine as therapeutics, where they can be used, for example, for the treatment and prophylaxis of the following illnesses: acute and chronic (in particular inflammatory and allergen-induced) airway disorders of varying origin (bronchitis, allergic bronchitis, bronchial asthma, emphysema, COPD); dermatoses (especially of proliferative, inflammatory and allergic type) such as psoriasis (vulgaris), toxic and allergic contact eczema, atopic eczema, seborrhoeic eczema, Lichen simplex, sunburn, pruritus in the anogenital area, alopecia greata, hypertrophic scars, discoid lupus erythematosus, follicular and widespread pyodermias, endogenous and exogenous acne, acne rosacea and other proliferative, inflammatory and allergic skin disorders; disorders which are based on an excessive release of TNF and leukotrienes, for example disorders of the arthritis type (rheumatoid arthritis, rheumatoid spondylitis, osteoarthritis and other arthritic conditions), disorders of the immune system (AIDS, multiple sclerosis), graft versus host reaction, allograft rejections, types of shock (septic shock, endotoxin shock, gram-negative sepsis, toxic shock syndrome and ARDS (adult respiratory distress syndrome)) and also generalized inflammations in the gastrointestinal region (Crohn's disease and ulcerative colitis); disorders which are based on allergic and/or chronic, immunological false reactions in the region of the upper airways (pharynx, nose) and the adjacent regions (paranasal sinuses, eyes), such as allergic rhinitis/sinusitis, chronic rhinitis/sinusitis, allergic conjunctivitis and also nasal polyps; but also disorders of the heart which can be treated by PDE inhibitors, such as cardiac insufficiency, or disorders which can be treated on account of the tissue-relaxant action of the PDE inhibitors, such as, for example, erectile dysfunction or colics of the kidneys and of the ureters in connection with kidney stones. In addition, the compounds of the invention are useful in the treatment of diabetes insipidus and conditions associated with cerebral metabolic inhibition, such as cerebral senility, senile dementia (Alzheimer's disease), memory impairment associated with Parkinson's disease or multi-infarct dementia; and also illnesses of the central nervous system, such as depressions or arteriosclerotic dementia; as well as for enhancing cognition. Yet in addition, the compounds of the invention are useful in the treatment of diabetes mellitus, leukaemia and osteoporosis.

The invention further relates to a method for the treatment of mammals, including humans, which are suffering from one of the above mentioned illnesses. The method is characterized in that a pharmacologically active and therapeutically effective and tolerable amount of one or more of the compounds according to the invention is administered to the ill mammal.

The invention further relates to the compounds according to the invention for use in the treatment and/or prophylaxis of illnesses, especially the illnesses mentioned.

The invention also relates to the use of the compounds according to the invention for the production of pharmaceutical compositions which are employed for the treatment and/or prophylaxis of the illnesses mentioned.

The invention also relates to the use of the compounds according to the invention for the production of pharmaceutical compositions for treating disorders which are mediated by phosphodiesterases, in particular PDE4-mediated disorders, such as, for example, those mentioned in the specification of this invention or those which are apparent or known to the skilled person.

The invention also relates to the use of the compounds according to the invention for the manufacture of pharmaceutical compositions having PDE4 inhibitory activity.

The invention furthermore relates to pharmaceutical compositions for the treatment and/or prophylaxis of the illnesses mentioned comprising one or more of the compounds according to the invention.

The invention yet furthermore relates to compositions comprising one or more compounds according to this invention and pharmaceutically acceptable auxiliaries and/or excipients.

The invention yet furthermore relates to compositions comprising one or more compounds according to this invention and a pharmaceutically acceptable carrier. Said compositions can be used in therapy, such as e.g. for treating, preventing or ameliorating one or more of the abovementioned diseases.

The invention still yet furthermore relates to pharmaceutical compositions according to this invention having PDE, particularly PDE4, inhibitory activity.

Additionally, the invention relates to an article of manufacture, which comprises packaging material and a pharmaceutical agent contained within said packaging material, wherein the pharmaceutical agent is therapeutically effective for antagonizing the effects of the cyclic nucleotide phosphodiesterase of type 4 (PDE4), ameliorating the symptoms of an PDE4-mediated disorder, and wherein the packaging material comprises a label or package insert which indicates that the pharmaceutical agent is useful for preventing or treating PDE4-mediated disorders, and wherein said pharmaceutical agent comprises one or more compounds of formula I according to the invention. The packaging material, label and package insert otherwise parallel or resemble what is generally regarded as standard packaging material, labels and package inserts for pharmaceuticals having related utilities.

The pharmaceutical compositions are prepared by processes which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, excipients, carriers, vehicles, diluents or adjuvants which are suitable for the desired pharmaceutical formulations on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

The administration of the pharmaceutical compositions according to the invention may be performed in any of the generally accepted modes of administration available in the art. Illustrative examples of suitable modes of administration include intravenous, oral, nasal, parenteral, topical, transdermal and rectal delivery. Oral delivery is preferred.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatoses, the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the active compounds is carried out in the order of magnitude customary for PDE inhibitors. Topical application forms (such as ointments) for the treatment of dermatoses thus contain the active compounds in a concentration of, for example, 0.1-99%. The dose for administration by inhalation is customarily between 0.01 and 3 mg per day. The customary dose in the case of systemic therapy (p.o. or i.v.) is between 0.003 and 3 mg/kg per day. In another embodiment, the dose for administration by inhalation is between 0.1 and 3 mg per day, and the dose in the case of systemic therapy (p.o. or i.v.) is between 0.03 and 3 mg/kg per day.

### Biological Investigations

The second messenger cyclic AMP (CAMP) is well-known for inhibiting inflammatory and immunocompetent cells. The PDE4 isoenzyme is broadly expressed in cells involved in the initiation and propagation of inflammatory diseases (H Tenor and C Schudt, in “Phosphodiesterase Inhibitors”, 21-40, “The Handbook of Immunopharmacology”, Academic Press, 1996), and its inhibition leads to an increase of the intracellular CAMP concentration and thus to the inhibition of cellular activation (J E Souness et al., Immunopharmacology 47:127-162, 2000).

The antiinflammatory potential of PDE4 inhibitors in vivo in various animal models has been described (M M Teixeira, TiPS 18: 164-170, 1997). For the investigation of PDE4 inhibition on the cellular level (in vitro), a large variety of proinflammatory responses can be measured. Examples are the superoxide production of neutrophilic (C Schudt et al., Arch Pharmacol 344: 682-690, 1991) or eosinophilic (A Hatzelmann et al., Brit J Pharmacol 114: 821-831, 1995) granulocytes, which can be measured as luminol-enhanced chemiluminescence, or the synthesis of tumor necrosis factor-α in monocytes, macrophages or dendritic cells (Gantner et al., Brit J Pharmacol 121: 221-231, 1997, and Pulmonary Pharmacol Therap 12: 377-386, 1999). In addition, the immunomodulatory potential of PDE4 inhibitors is evident from the inhibition of T-cell responses like cytokine synthesis or proliferation (DM Essayan, Biochem Pharmacol 57: 965-973, 1999). Substances which inhibit the secretion of the aforementioned proinflammatory mediators are those which inhibit PDE4. PDE4 inhibition by the compounds according to the invention is thus a central indicator for the suppression of inflammatory processes.

### Methods for Measuring Inhibition of PDE4 Activity

### Method a:

PDE4 activity was determined as described by Thompson et al. (Adv Cycl Nucl Res 10: 69-92, 1979) with some modifications (Bauer and Schwabe, Naunyn-Schmiedeberg's Arch Pharmacol 311: 193-198, 1980). At a final assay volume of 200 μl (96 well microtiter plates) the assay mixture contained 20 mM Tris (pH 7.4), 5 mM MgCl₂, 0.5 μM CAMP, [³H]cAMP (about 30,000 cpm/assay), the test compound and an aliquot of cytosol from human neutrophils which mainly contains PDE4 activity as described by Schudt et al. (Naunyn-Schmiedeberg's Arch Pharmacol 344: 682-690, 1991); the PDE3-specific inhibitor Motapizone (1 μM) was included to suppress PDE3 activity originating from contaminating platelets. Serial dilutions of the compounds were prepared in DMSO and further diluted 1:100 (v/v) in the assays to obtain the desired final concentrations of the inhibitors at a DMSO concentration of 1% (v/v) which by itself only slightly affected PDE4 activity.

After preincubation for 5 min at 37° C., the reaction was started by the addition of substrate (CAMP) and the assays were incubated for further 15 min at 37° C. 50 μl of 0.2 N HCl was added to stop the reaction and the assays were left on ice for about 10 min. Following incubation with 25 μg 5′-nucleotidase (Crotalus atrox snake venom) for 10 min at 37° C., the assays were loaded on QAE Sephadex A-25 (1 ml bed volume). The columns were eluted with 2 ml of 30 mM ammonium formiate (pH 6.0) and the eluate was counted for radioactivity. Results were corrected for blank values (measured in the presence of denatured protein) which were below 5% of total radioactivity. The amount of cyclic nucleotides hydrolyzed did not exceed 30% of the original substrate concentration. The IC₅₀-values for the compounds according to the invention for the inhibition of the PDE4 activity were determined from the concentration-inhibition curves by nonlinear-regression.

### Method b:

The PDE4B2 (GB no. M97515) was a gift of Prof. M. Conti (Stanford University, USA). It was amplified from the original plasmid (pCMV5) via PCR with primers Rb9 (5′-GCCAGCGTGCAAATAATGMGG-3′) and Rb10 (5′-AGAGGGGGATTATGTATCCAC-3′) and cloned into the pCR-Bac vector (Invitrogen, Groningen, NL).

The recombinant baculovirus was prepared by means of homologous recombination in SF9 insect cells. The expression plasmid was cotransfected with Bac-N-Blue (Invitrogen, Groningen, NL) or Baculo-Gold DNA (Pharmingen, Hamburg) using a standard protocol (Pharmingen, Hamburg). Wt virus-free recombinant virus supernatant was selected using plaque assay methods. After that, high-titre virus supernatant was prepared by amplifying 3 times. PDE was expressed in SF21 cells by infecting 2×10⁶ cells/ml with an MOI (multiplicity of infection) between 1 and 10 in serum-free SF900 medium (Life Technologies, Paisley, UK). The cells were cultured at 28° C. for 48-72 hours, after which they were pelleted for 5-10 min at 1000 g and 4° C.

The SF21 insect cells were resuspended, at a concentration of approx. 10⁷ cells/ml, in ice-cold (4° C.) homogenization buffer (20 mM Tris, pH 8.2, containing the following additions: 140 mM NaCl, 3.8 mM KCl, 1 mM EGTA, 1 mM MgCl₂, 10 mM β-mercaptoethanol, 2 mM benzamidine, 0.4 mM Pefablock, 10 μM leupeptin, 10 μM pepstatin A, 5 μM trypsin inhibitor) and disrupted by ultrasonication. The homogenate was then centrifuged for 10 min at 1000×g and the supernatant was stored at −80° C. until subsequent use (see below). The protein content was determined by the Bradford method (BioRad, Munich) using BSA as the standard.

PDE4B2 activity is inhibited by the said compounds in a modified SPA (scintillation proximity assay) test, supplied by Amersham Biosciences (see procedural instructions “phosphodiesterase [3H]cAMP SPA enzyme assay, code TRKQ 7090”), carried out in 96-well microtitre plates (MTP's). The test volume is 100 μl and contains 20 mM Tris buffer (pH 7.4), 0.1 mg of BSA (bovine serum albumin)/ml, 5 mM Mg²⁺, 0.5 μM CAMP (including about 50,000 cpm of [3H]cAMP), 1 μl of the respective substance dilution in DMSO and sufficient recombinant PDE (1000×g supernatant, see above) to ensure that 10-20% of the CAMP is converted under the said experimental conditions. The final concentration of DMSO in the assay (1% v/v) does not substantially affect the activity of the PDE investigated. After a preincubation of 5 min at 37° C., the reaction is started by adding the substrate (CAMP) and the assay is incubated for a further 15 min; after that, it is stopped by adding SPA beads (50 μl). In accordance with the manufacturers instructions, the SPA beads had previously been resuspended in water, but were then diluted 1:3 (v/v) in water; the diluted solution also contains 3 mM IBMX to ensure a complete PDE activity stop. After the beads have been sedimented (>30 min), the MTP's are analyzed in commercially available luminescence detection devices. The corresponding IC₅₀ values of the compounds for the inhibition of PDE activity are determined from the concentration-effect curves by means of non-linear regression.

The inhibitory values determined for the compounds according to the invention follow from the following table A, in which the numbers of the compounds correspond to the numbers of the examples.

The inhibitory values of the compounds 1 to 19 have been determined according to Method a. The inhibitory values of the compounds 20 to 24 and 26 to 29 have been determined according to Method b.

TABLE A
Inhibition of the PDE4 activity

| Compound | −log IC₅₀ |
|---|---|
| 1 to 19, 20 to | The inhibitory |
| 24, and 26 to | values are in |
| 31 | the range |
| | from 7.36 to |
| | 9.33 |

1. A compound of formula I, 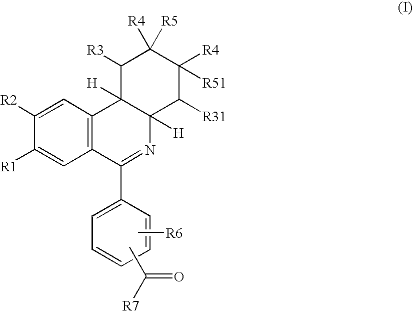
in which
R1 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
R2 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
or in which R1 and R2 together are a 1-2C-alkylenedioxy group,
R3 is hydrogen or 1-4C-alkyl,
R31 is hydrogen or 1-4C-alkyl,
or in which R3 and R31 together are a 1-4C-alkylene group,
R4 is hydrogen or 1-4C-alkyl,
R5 is hydrogen,
R51 is hydrogen,
or in which R5 and R51 together represent an additional bond,
R6 is hydrogen, halogen, 1-4C-alkyl or 1-4C-alkoxy,
wherein either,
R7 is —N(R8)R9, in which
R8 is hydrogen, 1-4C-alkyl or 1-4C-alkoxy-2-4C-alkyl,
R9 is hydrogen, 1-4C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
Har1 is optionally substituted by R91 and/or R92, and is a 5- to 10-membered monocyclic or fused bicyclic unsaturated heteroaryl radical comprising 1 to 4 heteroatoms independently selected from the group consisting of oxygen, nitrogen and sulfur, in which
R91 is 1-4C-alkyl or 1-4C-alkoxy,
R92 is 1-4C-alkyl or 1-4C-alkoxy,
R93 is hydrogen or 1-4C-alkyl,
R94 is hydrogen or 1-4C-alkyl,
or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
or R8 and R9 together and with inclusion of the nitrogen atom, to which they are attached, form a heterocyclic ring Het2, in which
Het2 is optionally substituted by R10, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is hydrogen or 1-4C-alkyl,
R13 is hydrogen or 1-4C-alkyl,
or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
or,
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is optionally substituted by R201 and/or R202, and is a 5- to 10-membered monocyclic or fused bicyclic unsaturated heteroaryl radical comprising 1 to 4 heteroatoms independently selected from the group consisting of oxygen, nitrogen and sulfur, in which
R201 is 1-4C-alkyl or 1-4C-alkoxy,
R202 is 1-4C-alkyl or 1-4C-alkoxy,
Het6 is optionally substituted by R203 and/or R204, and is a monocyclic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy
R206 is 1-4C-alkoxy
R21 is aminosulphonyl,
or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which R18 is 1-4C-alkyl,
under the provisio, that those compounds, in which
R1 is hydroxyl, 1-4C-alkoxy, 3-7C-cycloalkoxy, 3-7C-cycloalkylmethoxy, or completely or predominantly fluorine-substituted 1-4C-alkoxy,
R2 is hydroxyl, 1-4C-alkoxy, 3-7C-cycloalkoxy, 3-7C-cycloalkylmethoxy, or completely or predominantly fluorine-substituted 1-4C-alkoxy,
or in which R1 and R2 together are a 1-2C-alkylenedioxy group,
and
R7 is —N(R8)R9, in which
either
R8 is hydrogen or 1-4C-alkyl, and
R9 is hydrogen, 1-4C-alkyl or 3-7C-cycloalkyl,
or
R8 is hydrogen or 1-4C-alkyl, and
R9 is pyridyl optionally substituted by 1-4C-alkyl or 1-4C-alkoxy,
or
or R8 and R9 together and with inclusion of the nitrogen atom, to which they are attached, form a heterocyclic ring Het2, in which
Het2 is pyrrolidin-1-yl, piperidin-1-yl, piperazin-1-yl, 4-methyl-piperazin-1-yl or morpholin-4-yl,
are thereof disclaimed,
or a salt, N-oxide or salt of an N-oxide thereof.

2. A compound of formula I according to claim 1 in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3, R31, R4, R5, R51 and R6 are hydrogen,
wherein either,
R7 is —N(R8)R9, in which
R8 is hydrogen, 1-4C-alkyl or 1-4C-alkoxy-2-4C-alkyl,
R9 is hydrogen, 1-4C-alkyl, mono- or di-1-4C-alkoxy-2-4C-alkyl, hydroxy-2-4C-alkyl, mono- or di-1-4C-alkoxycarbonyl-1-4C-alkyl, Har1, pyridinyl-1-4C-alkyl, 3-7C-cycloalkyl, or 2-4C-alkyl substituted by —NR(93)R94, in which
either
Har1 is optionally substituted by R91 and/or R92, and is a 9- or 10-membered fused bicyclic unsaturated heteroaryl radical comprising 1 to 4 heteroatoms independently selected from the group consisting of oxygen, nitrogen and sulfur, in which
R91 is 1-4C-alkyl,
R92 is 1-4C-alkyl,
or
Har1 is optionally substituted by R91 and/or R92, and is a 6-membered monocyclic unsaturated heteroaryl radical comprising one or two nitrogen atoms, in which
R91 is 1-4C-alkoxy,
R92 is 1-4C-alkoxy,
R93 is hydrogen or 1-4C-alkyl,
R94 is hydrogen or 1-4C-alkyl,
or R93 and R94 together and with inclusion of the nitrogen atom, to which they are attached, form a heterocyclic ring Het1, in which
Het1 is optionally substituted by R931, and is a 3- to 7-membered saturated monocyclic heterocyclic ring radical comprising the nitrogen atom, to which R93 and R94 are bonded, and optionally one further heteroatom selected from the group consisting of oxygen, nitrogen and sulfur, in which
R931 is 1-4C-alkyl,
or R8 and R9 together and with inclusion of the nitrogen atom, to which they are attached, form a heterocyclic ring Het2, in which
Het2 is optionally substituted by R10, and is a 3- to 7-membered saturated monocyclic heterocyclic ring radical comprising the nitrogen atom, to which R8 and R9 are bonded, and optionally one further heteroatom selected from the group consisting of oxygen, nitrogen and sulfur, in which
R10 is 1-4C-alkyl, —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is hydrogen or 1-4C-alkyl,
R13 is hydrogen or 1-4C-alkyl,
or R12 and R13 together and with inclusion of the nitrogen atom, to which they are attached, form a heterocyclic ring Het3, in which
Het3 is optionally substituted by R121, and is a 3- to 7-membered saturated monocyclic heterocyclic ring radical comprising the nitrogen atom, to which R12 and R13 are bonded, and optionally one further heteroatom selected from the group consisting of oxygen, nitrogen and sulfur, in which
R121 is 1-4C-alkyl,
R14 is hydrogen or 1-4C-alkyl,
R15 is hydrogen or 1-4C-alkyl,
or R14 and R15 together and with inclusion of the nitrogen atom, to which they are attached, form a heterocyclic ring Het4, in which
Het4 is optionally substituted by R141, and is a 3- to 7-membered saturated monocyclic heterocyclic ring radical comprising the nitrogen atom, to which R14 and R15 are bonded, and optionally one further heteroatom selected from the group consisting of oxygen, nitrogen and sulfur, in which
R141 is 1-4C-alkyl,
R16 is hydrogen, 1-4C-alkyl or pyridyl,
R17 is hydrogen or 1-4C-alkyl,
or R16 and R17 together and with inclusion of the nitrogen atom, to which they are attached, form a heterocyclic ring Het5, in which
Het5 is optionally substituted by R161, and is a 3- to 7-membered saturated monocyclic heterocyclic ring radical comprising the nitrogen atom, to which R16 and R17 are bonded, and optionally one further heteroatom selected from the group consisting of oxygen, nitrogen and sulfur, in which
R141 is 1-4C-alkyl,
or,
R7 is —NH—N(R18)R19, in which
R18 is hydrogen,
R19 is —C(O)R20, or R21-substituted phenyl, in which
R20 is Har2, Het6, or Aryl-1-4C-alkyl, in which
Har2 is a 6-membered monocyclic unsaturated heteroaryl radical comprising one or two nitrogen atoms,
Het6 is optionally substituted by R203 and/or R204, and is a monocyclic 3- to 7-membered saturated heterocyclic ring radical comprising one to three heteroatoms, each of which is selected from the group consisting of nitrogen, oxygen and sulfur, in which
R203 is 1-4C-alkyl,
R204 is 1-4C-alkyl,
Aryl is R205- and/or R206-substituted phenyl,
R205 is 1-4C-alkoxy,
R206 is 1-4C-alkoxy,
R21 is aminosulphonyl,
or R18 and R19 together and with inclusion of the nitrogen atom, to which they are attached, form a heterocyclic ring Het7, in which
Het7 is optionally substituted by R181, and is a 3- to 7-membered saturated monocyclic heterocyclic ring radical comprising the nitrogen atom, to which R18 and R19 are bonded, and optionally one further heteroatom selected from the group consisting of oxygen, nitrogen and sulfur, in which
R181 is 1-4C-alkyl,
under the provisio, that those compounds, in which
R1 is 1-2C-alkoxy, or completely or predominantly fluorine-substituted 1-2C-alkoxy,
R2 is 1-2C-alkoxy, or completely or predominantly fluorine-substituted 1-2C-alkoxy,
and
R7 is —N(R8)R9, in which
either
R8 is hydrogen or 1-4C-alkyl, and
R9 is hydrogen, 1-4C-alkyl or 3-7C-cycloalkyl,
or
R8 is hydrogen or 1-4C-alkyl, and
R9 is pyridyl optionally substituted by 1-4C-alkoxy,
or
or R8 and R9 together and with inclusion of the nitrogen atom, to which they are attached, form a heterocyclic ring Het2, in which
Het2 is pyrrolidin-1-yl, piperidin-1-yl, piperazin-1-yl, 4-methyl-piperazin-1-yl or morpholin-4-yl,
are thereof disclaimed,
or a salt, N-oxide or salt of an N-oxide thereof.

3. A compound of formula I according to claim 1 in which
one of R1 and R2 is methoxy, and the other is methoxy, ethoxy, 2,2-difluoroethoxy, or difluoromethoxy,
R3, R31, R4, R5, R51 and R6 are hydrogen,
wherein either,
R7 is —N(R8)R9, in which
R8 is hydrogen, 1-4C-alkyl or 1-4C-alkoxy-2-4C-alkyl,
R9 is 1-4C-alkyl, mono- or di-1-4C-alkoxy-2-4C-alkyl, hydroxy-2-4C-alkyl, mono- or di-1-2C-alkoxycarbonyl-1-4C-alkyl, Har1, pyridinyl-1-4C-alkyl, 3-5C-cycloalkyl, or 2-4C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl,
R93 and R94 together and with inclusion of the nitrogen atom, to which they are attached, form a heterocyclic ring Het1, in which
Het1 is morpholinyl or 4N-(1-4C-alkyl)-piperazinyl,
or R8 and R9 together and with inclusion of the nitrogen atom, to which they are attached, form a heterocyclic ring Het2, in which
Het2 is pyrrolidinyl, morpholinyl or 4N—(R10)-piperazinyl, in which
R10 is —C(O)R11, pyridyl, 2-4C-alkyl substituted by —NR(14)R15, or 1-4C-alkyl substituted by —C(O)N(R16)R17, in which
R11 is 1-4C-alkyl substituted by —NR(12)R13, in which
R12 is 1-4C-alkyl,
R13 is 1-4C-alkyl,
or R12 and R13 together and with inclusion of the nitrogen atom, to which they are attached, form a heterocyclic ring Het3, in which
Het3 is morpholinyl,
R14 is 1-4C-alkyl,
R15 is 1-4C-alkyl,
or R14 and R15 together and with inclusion of the nitrogen atom, to which they are attached, form a heterocyclic ring Het4, in which
Het4 is morpholinyl,
R16 is 1-4C-alkyl or pyridyl,
R17 is hydrogen or 1-4C-alkyl,
or R16 and R17 together and with inclusion of the nitrogen atom, to which they are attached, form a heterocyclic ring Het5, in which
Het5 is pyrrolidinyl or morpholinyl,
or,
R7 is —NH—N(R18)R19, in which
R18 is hydrogen,
R19 is —C(O)R20, or R21-substituted phenyl, in which
R20 is pyridinyl, morpholinyl, 1N—(R203)-4N—(R204)-piperazinyl, or Aryl-1-2C-alkyl, in which
R203 is 1-4C-alkyl,
R204 is 1-4C-alkyl,
Aryl is 3,4-dimethoxyphenyl,
R21 is aminosulphonyl,
or R18 and R19 together and with inclusion of the nitrogen atom, to which they are attached, form a heterocyclic ring Het7, in which
Het7 is morpholinyl or 4N—(R181)-piperazinyl, in which
R181 is 1-4C-alkyl,
under the provisio, that those compounds, in which
one of R1 and R2 is methoxy, and the other is methoxy, ethoxy or difluoromethoxy,
and
R7 is —N(R8)R9, in which
either
R8 is hydrogen or 1-4C-alkyl, and
R9 is 1-4C-alkyl or 3-5C-cycloalkyl,
or
or R8 and R9 together and with inclusion of the nitrogen atom, to which they are attached, form a heterocyclic ring Het2, in which
Het2 is pyrrolidin-1-yl or morpholin-4-yl,
are thereof disclaimed,
or a salt, N-oxide or salt of an N-oxide thereof.

4. A compound of formula I according to claim 1 in which
R1 is methoxy,
R2 is methoxy, ethoxy, 2,2-difluoroethoxy, or difluoromethoxy,
R3, R31, R4, R5, R51 and R6 are hydrogen,
wherein either,
R7 is —N(R8)R9, in which
R8 is hydrogen, methyl, ethyl, isopropyl or 2-methoxyethyl,
R9 is methoxy-2-3C-alkyl, 2,2-diethoxyethyl, 3-hydroxy-propyl, methoxycarbonylmethyl, 1,2-di-(methoxycarbonyl)-ethyl, Har1, 2-pyridinyl-ethyl, or 2-3C-alkyl substituted by —NR(93)R94, in which
Har1 is 2,6-dimethoxypyridinyl, quinolinyl, 2,3-dimethyl-imidazo[1,2-a]pyridinyl or [1,7]naphthyridinyl,
R93 and R94 together and with inclusion of the nitrogen atom, to which they are attached, form a heterocyclic ring Het1, in which
Het1 is morpholinyl or 4N-(1-2C-alkyl)-piperazinyl,
or R8 and R9 together and with inclusion of the nitrogen atom, to which they are attached, form a heterocyclic ring Het2, in which
Het2 is 4N—(R10)-piperazinyl, in which
R10 is —C(O)R1, pyridyl, ethyl substituted by —NR(14)R15, or methyl substituted by —C(O)N(R16)R17, in which
R11 is methyl substituted by —NR(12)R13, in which
R12 is methyl,
R13 is methyl,
or R12 and R13 together and with inclusion of the nitrogen atom, to which they are attached, form a heterocyclic ring Het3, in which
Het3 is morpholinyl,
R14 is methyl,
R15 is methyl,
or R14 and R15 together and with inclusion of the nitrogen atom, to which they are attached, form a heterocyclic ring Het4, in which
Het4 is morpholinyl,
R16 is methyl or pyridyl,
R17 is hydrogen or methyl,
or R16 and R17 together and with inclusion of the nitrogen atom, to which they are attached, form a heterocyclic ring Het5, in which
Het5 is pyrrolidinyl or morpholinyl,
or,
R7 is —NH—N(R18)R19, in which
R18 is hydrogen,
R19 is —C(O)R20, or R21-substituted phenyl, in which
R20 is pyridinyl, morpholin-4-yl, 1N—(R203)-4N—(R204)-piperazinyl, or Aryl-methyl, in which
R203 is methyl,
R204 is methyl,
Aryl is 3,4-dimethoxyphenyl,
R21 is aminosulphonyl,
or R18 and R19 together and with inclusion of the nitrogen atom, to which they are attached, form a heterocyclic ring Het7, in which
Het7 is morpholinyl or 4N—(R181)-piperazinyl, in which
R181 is methyl,
whereby —C(O)R7 is bonded in the meta or para position with respect to the binding position, in which the phenyl ring is attached to the phenanthridine ring system,
or a salt, N-oxide or salt of an N-oxide thereof.

5. A compound of formula I as claimed in claim 1 in which
R1 is methoxy,
R2 is ethoxy, 2,2-difluoroethoxy or difluoromethoxy,
R3, R31, R4, R5, R51 and R6 are hydrogen,
R7 is —N(R8)R9, in which
either
R8 is hydrogen, and
R9 is cyclopropyl or cyclobutyl,
or
R8 is isopropyl, and
R9 is isopropyl,
whereby —C(O)R7 is bonded in the meta or para position with respect to the binding position, in which the phenyl ring is attached to the phenanthridine ring system,
or a salt, N-oxide or salt of an N-oxide thereof.

6. A compound according to claim 1, which has, with respect to the positions 4a and 10b, the configuration shown in formula I*: 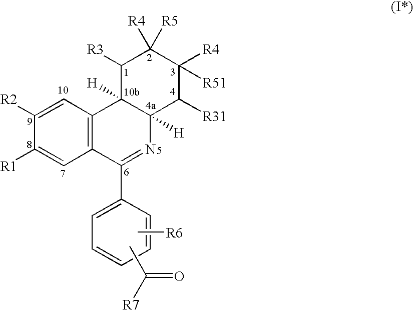
or a salt, N-oxide or salt of an N-oxide thereof.

7. (canceled)

8. A pharmaceutical composition comprising one or more compounds as claimed in claim 1, or a pharmaceutically acceptable salt, N-oxide or salt of an N-oxide thereof, together with a pharmaceutical auxiliary and/or excipient.

9. (canceled)

10. A method for treating an illness in a patient comprising administering to said patient a therapeutically effective amount of a compound as claimed in claim 1, or a pharmaceutically acceptable salt, N-oxide or salt of an N-oxide thereof.

11. A method for treating an airway disorder in a patient comprising administering to said patient a therapeutically effective amount of a compound as claimed in claim 1, or a pharmaceutically acceptable salt, N-oxide or salt of an N-oxide thereof.
